(19) United States
(12) Patent Application Publication
(10) Pub. No.: US 2025/0136664 A1
(43) Pub. Date: May 1, 2025

(54) SWITCH RECEPTORS USING IL-9 SIGNALING DOMAINS

(71) Applicants: Parker Institute for Cancer Immunotherapy, San Francisco, CA (US); The Board of Trustees of the Leland Stanford Junior University, Stanford, CA (US)

(72) Inventors: John CONNOLLY, Red Oak, TX (US); Sean PARKER, San Francisco, CA (US); Kenan Christopher GARCIA, San Francisco, CA (US)

(21) Appl. No.: 19/003,599

(22) Filed: Dec. 27, 2024

Related U.S. Application Data
(62) Division of application No. 18/328,473, filed on Jun. 2, 2023, now Pat. No. 12,221,469.
Application No. 18/328,473 is a continuation of application No. 17/946,894, filed on Sep. 16, 2022, now abandoned.
(60) Provisional application No. 63/245,661, filed on Sep. 17, 2021.

(51) Int. Cl. C07K 14/715; A61K 40/11; A61K 40/31; A61K 40/42; C07K 14/705; C07K 14/71; C12N 5/078; C12N 5/0783; C12N 15/63; C12N 15/85

(52) U.S. Cl. CPC C07K 14/7155; C07K 14/70578; A61K 40/11; A61K 40/31; A61K 40/4205; A61K 40/4217; A61K 40/4261; A61K 2239/23; A61K 2239/53; A61K 2239/59; C07K 14/70503; C07K 14/70521; C07K 14/71; C07K 2319/03; C07K 2319/32; C12N 5/0634; C12N 5/0636; C12N 15/63; C12N 15/85

(57) ABSTRACT

The present disclosure generally relates to, inter alia, a class of chimeric switch receptors containing an endodomain of an IL-9 receptor, engineered to modulate transcriptional regulation in a ligand-dependent manner. The disclosure also provides compositions and methods useful for producing such receptors, nucleic acids encoding same, host cells genetically modified with the nucleic acids, as well as methods for modulating gene expression, modulating an activity of a cell, and/or for the treatment of various health conditions or diseases.

### CROSS-REFERENCE TO RELATED APPLICATIONS

This application is a divisional application of 18/328,473, filed Jun. 2, 2023, which is a continuation of U.S. patent application Ser. No. 17/946,894, filed Sep. 16, 2022, which claims the benefit of U.S. Provisional Patent Application No. 63/245,661, filed Sep. 17, 2021, the disclosures of which are incorporated by reference herein in their entireties.

### STATEMENT AS TO RIGHTS TO INVENTIONS MADE UNDER FEDERALLY SPONSORED RESEARCH AND DEVELOPMENT

This invention was made with Government support under Contract AI051321 awarded by the National Institutes of Health. The Government has certain rights in the invention.

### REFERENCE TO AN ELECTRONIC SEQUENCE LISTING

The contents of the electronic sequence listing 051288-502001US.xml; Size: 348 Kb; and Date of Creation: Dec. 4, 2022 is hereby incorporated by reference in its entirety.

### FIELD

The present disclosure relates generally to synthetic cellular receptors that bind extracellular ligands and have IL-9 endodomains. The disclosure also provides compositions and methods useful for producing such receptors, nucleic acids encoding same, host cells genetically modified with the nucleic acids, as well as methods for modulating gene expression, modulating an activity of a cell, and/or for the treatment of various health conditions or diseases.

### BACKGROUND

The manipulation of cells, particularly immune cells, to differentiate, develop specialized functions and expand in numbers is of great clinical interest. Many protein factors that affect these activities are known in the art, including in particular cytokines and chemokines. However, these signaling molecules also have pleiotropic effects on cells not targeted for manipulation, and thus methods of selectively activating signaling in a targeted cell population are desirable. The ability to engineer immune cells to carry out controlled behaviors is of interest in the field. For example, in adoptive immunotherapy T cells are isolated from blood, processed ex vivo, and re-infused into patients. Such T cells have been developed for use in therapeutic applications such as the treatment of cancer, infection, and autoimmune diseases.

A critical challenge in cell based therapies is the ability to engineer receptors that respond to native molecules while also allowing the selective manipulation of immune cells. Some groups have manipulated proteins to bind and respond to modified ligands in a manner independent, or orthogonal, from the influence of the native proteins or ligands. This technology relies on the engineering of both an orthogonal cytokine and an orthogonal receptor, and, relies on the premise that native molecules will not recognize the orthogonal receptor. One of the challenges associated with generating orthogonal ligand-receptor pairs is finding mutations that efficiently prevent activation by the endogenous molecule without otherwise compromising the receptor's structure or intrinsic ability to activate gene transcription. In alternative methods, to modulate signaling in immune cells, the only approach to prevent negative signals delivered by molecules such as PD-1, is to give patients systemic treatment of antagonistic antibodies that bind to PD-1. This approach has the limitation that systemic treatment prevents T cells that are present in the tumor microenvironment and the entire immune system from being inactivated, which in some patients can result in autoimmunity or systemic inflammatory syndrome (Beck et al., 2006, J Clin Oncol 24:2283-9; Blansfield et al., 2005, J Immunother 28:593-8; Dougan et al., 2009, Annual Review of Immunology 27:83-117).

The disclosure provided here provides solutions to the problems existing with previous attempts to manipulate immune cells and potentially offer improved methods for treatments involving cell transfer.

### SUMMARY

The present disclosure generally relates to, among other things, chimeric switch receptors containing an endodomain of an IL-9 receptor, engineered to modulate transcriptional regulation in a ligand-dependent manner. The activity of these switch receptors can be controlled by the presence of an extracellular ligand, allowing for spatial and temporal control of specific gene expression in mammalian cells, as well as for use in modulating cell activities or in treating various health conditions, such as diseases.

In one aspect, provided herein are recombinant nucleic acid molecules encoding chimeric receptors that comprise an extracellular portion comprising a binding domain of an endogenous cytokine receptor, an intracellular portion comprising an endodomain of an IL-9 receptor, and a transmembrane domain that joins the extracellular portion and intracellular portion.

In some embodiments, the recombinant nucleic acid molecule further comprises one or more linkers.

In some embodiments, the endogenous cytokine receptor is selected from IL-2rb, IL-2ra, IL-4r, IL-7ra, IL-15ra, and IL-21ra.

In some embodiments, the endogenous cytokine receptor comprises an amino acid sequence having at least 80% sequence identity to an amino acid sequence selected from SEQ ID Nos: 1-6.

In some embodiments, the transmembrane domain is selected from the transmembrane domain of IL-9, IL-7ra, IL-2rb, and TNFR1. In some embodiments, the transmembrane domain comprises an amino acid sequence selected from SEQ ID Nos: 53-56.

In some embodiments, the chimeric receptor comprises an amino acid sequence having at least 80% sequence identity to an amino acid sequence selected from SEQ ID Nos: 63-80.

Another aspect provides recombinant nucleic acid molecules encoding chimeric receptors comprising an extracellular portion comprising a binding domain of an endogenous inhibitory receptor an intracellular portion comprising an endodomain of an IL-9 receptor linked to a BOX½ common gamma chain domain; and a transmembrane domain that joins the extracellular portion and the intracellular portion.

In some embodiments, the recombinant nucleic acid molecule further comprises one or more linkers.

In some embodiments, the endogenous inhibitory receptor is selected from TGF-beta R1, TGF-beta R2, IL-10ra, FAS, CTLA4, LAG3, TIM3, PD1, ILT2, ILT3, ILT4, ILT5, and VEGF.

In some embodiments, the endogenous inhibitory receptor comprises an amino acid sequence having at least 80% sequence identity to an amino acid sequence selected from SEQ ID Nos: 7-52.

In some embodiments, the transmembrane domain is selected from the transmembrane domain of IL-9, IL-7ra, IL-2rb, and TNER1.

In some embodiments, the transmembrane domain is selected from the transmembrane domain of IL-9, IL-7ra, IL-2rb, and TNER1.

In some embodiments, the chimeric receptor comprises an amino acid sequence having at least 80% sequence identity to an amino acid sequence selected from SEQ ID Nos: 81-203.

Another aspect provides recombinant nucleic acid molecules encoding chimeric receptors comprising an extracellular portion comprising a binding domain of an endogenous inhibitory receptor linked to an agent specific for the common gamma chain; an intracellular portion comprising an endodomain of an IL-9 receptor; and a transmembrane domain that joins the extracellular portion and the intracellular portion.

In some embodiments, the recombinant nucleic acid molecule further comprises one or more linkers.

In some embodiments, the endogenous inhibitory receptor is selected from TGF-beta R1, TGF-beta R2, IL-10ra, FAS, CTLA4, LAG3, TIM3, PD1, ILT2, ILT3, ILT4, ILT5, and VEGF.

In some embodiments, the endogenous inhibitory receptor comprises an amino acid sequence having at least 80% sequence identity to an amino acid sequence selected from SEQ ID Nos: 7-52.

In some embodiments, the transmembrane domain is selected from the transmembrane domain of IL-9, IL-7ra, IL-2rb, and TNFR1. In some embodiments, the transmembrane domain comprises an amino acid sequence selected from SEQ ID Nos: 53-56.

In some embodiments, the agent specific for the common gamma chain comprises a nanobody, a darpin, IL-2, IL-4, IL-7, and scFV.

Another aspect relates to an expression vector comprising the recombinant nucleic acid molecules of the disclosure.

Another aspect relates to a host cell comprising the expression vector of the present disclosure. In some embodiments, the host cell is a mammalian cell. In some embodiments, the host cell is an immune cell.

Another aspect provides a composition comprising a recombinant nucleic acid of the present disclosure.

Another aspect provides a composition of cells comprising the expression vector of the present disclosure.

Another aspect provides a polypeptide encoded by the recombinant nucleic acid the present disclosure.

Another aspect provides a composition of one or more polypeptides encoded by one or more recombinant nucleic acids of the present disclosure.

Another aspect provides a composition of cells capable of expressing the chimeric receptor encoded by the recombinant nucleic acid of the present disclosure.

Another aspect provides a composition of cells comprising a chimeric receptor comprising an amino acid sequence selected from SEQ ID Nos: 63-203.

Another aspect relates to a method for modulating the activity of an immune cell comprising administering, to an immune cell, the recombinant nucleic acid of the present disclosure.

Another aspect relates to a method of treating a subject that involves administering, to the subject, a cell expressing the recombinant nucleic acid of the present disclosure.

In some embodiments, the subject is treated for cancer.

In some embodiments, the subject is treated for autoimmune disease.

In some embodiments, the subject is treated for infection.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows the pSTAT expression profile in either stimulated or unstimulated primary human T cells transduced with a lentiviral vector encoding switch receptors of SEQ ID NO: 63+CAR+(IL21R ECD+IL9R TM+IL9R ICD and CAR 4D5), SEQ ID NO:66 (IL15Ra+IL9R TM+IL9R ICD), SEQ ID NO:72+CAR+(IL4R ECD+IL9R TM+IL9R ICD and CAR 4D5), or SEQ ID NO:153+CAR+(IL10Ra ECD+IL9R TM+IL9R ICD and CAR 4D5).

FIG. 2 shows a real-time cytotoxicity assay (RTCA) with T cells co-expressing switch receptor SEQ63 (IL21R ECD+IL9R TM+IL9R ICD) and CAR (4D5) against SKOV-3 human ovarian adenocarcinoma cells expressing HER2. Double-positive T cells (SEQ63+CAR+) were left unstimulated (“no stim”) before being added on SKOV-3 tumor cells at a 1:8 effector-to-target ratio, or preconditioned for 48 hours with IL21 before addition to the plate with continued ligand stimulation (“preconditioned+IL21”). Untransduced T cells (UTD) served as control and were added on tumor cells with continued IL21 stimulation (“UTD+IL21”).

FIG. 3 shows a real-time cytotoxicity assay (RTCA) with T cells co-expressing switch receptor SEQ72 (IL4R ECD+IL9R TM+IL9R ICD) and CAR (4D5) against SKOV-3 human ovarian adenocarcinoma cells expressing HER2. Double-positive T cells (SEQ72+CAR+) were left unstimulated (“no stim”) before being added on SKOV-3 tumor cells at a 1:8 effector-to-target ratio, or preconditioned for 48 hours with IL4 before addition to the plate with continued ligand stimulation (“preconditioned+IL4”). Untransduced T cells (UTD) served as control and were added on tumor cells with continued IL4 stimulation (“UTD+IL4”).

FIG. 4 shows a real-time cytotoxicity assay (RTCA) with T cells co-expressing switch receptor SEQ113 (Fas ECD+IL9R TM+IL9R ICD) and CAR (4D5) against SKOV-3 human ovarian adenocarcinoma cells expressing HER2. Double-positive T cells (SEQ113+CAR+) were left unstimulated (“no stim”) before being added on SKOV-3 tumor cells at a 1:4 effector-to-target ratio, or preconditioned for 48 hours with FasL before addition to the plate (“preconditioned+no stim”). Untransduced T cells (UTD) served as control and were added on tumor cells unstimulated (“UTD+no stim”).

FIG. 5 shows hybrid cytokine receptors induce functional activation of T cells in response to ligand stimulation. T cells were transduced with hybrid cytokine receptor SEQ63 (IL21R ECD+IL9R TM+IL9R ICD) and CAR (4D5) and cocultured with IL-21 (i) before, (ii) during, or (iii) before+during, co-culture with target cells for 160 hrs. Cell culture supernatants were collected and the concentrations of effector cytokines measured by Luminex assay. The graph depicts fold change over unstimulated.

FIG. 6 shows hybrid cytokine receptors induce functional activation of T cells in response to ligand stimulation. T cells were transduced with hybrid cytokine receptor SEQ72 (IL4R ECD+IL9R TM+IL9R ICD) and CAR (4D5) and cocultured with IL-4 (i) before, (ii) during, or (iii) before+during, co-culture with target cells for 160 hrs. Cell culture supernatants were collected and the concentrations of effector cytokines measured by Luminex assay. The graph depicts fold change over unstimulated.

FIG. 7 shows hybrid cytokine receptors induce functional activation of T cells in response to ligand stimulation. T cells were transduced with hybrid cytokine receptor SEQ72 (IL4R ECD+IL9R TM+IL9R ICD) and CAR (4D5) and cocultured with IL-4 (i) before, (ii) during, or (iii) before+during, co-culture with target cells for 160 hrs. Cell culture supernatants were collected and the concentrations of effector cytokines measured by Luminex assay. The graphs shows concentration of EGF in pg/mL.

FIGS. 8A-8B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 8A) and SEQ72 (IL4R ECD+IL9R TM+IL9R ICD) and CAR (4D5) (FIG. 8B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of FGF-2 in pg/mL.

FIGS. 9A-9B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 9A) and SEQ72 (IL4R ECD+IL9R TM+IL9R ICD) and CAR (4D5) (FIG. 9B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of GM-CSF in pg/mL.

FIG. 10 shows hybrid cytokine receptors induce functional activation of T cells in response to ligand stimulation. T cells were transduced with hybrid cytokine receptor SEQ72 (IL4R ECD+IL9R TM+IL9R ICD) and CAR (4D5) and cocultured with its respective ligand (i) before, (ii) during, or (iii) before+during, co-culture with target cells for 160 hrs. Cell culture supernatants were collected and the concentrations of effector cytokines measured by Luminex assay. The graphs shows concentration of IFNa2 in pg/mL.

FIGS. 11A-11B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 11A) and SEQ72 (IL4R ECD+IL9R TM+IL9R ICD) and CAR (4D5) (FIG. 11B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of IFNg in pg/mL.

FIG. 12 shows hybrid cytokine receptors induce functional activation of T cells in response to ligand stimulation. T cells were transduced with hybrid cytokine receptor SEQ63 (IL21R ECD+IL9R TM+IL9R ICD) and CAR (4D5) and cocultured with its respective ligand (i) before, (ii) during, or (iii) before+during, co-culture with target cells for 160 hrs. Cell culture supernatants were collected and the concentrations of effector cytokines measured by Luminex assay. The graphs shows concentration of IL-10 in pg/mL.

FIGS. 13A-13B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 13A) and SEQ72 (IL4R ECD+IL9R TM+IL9R ICD) and CAR (4D5) (FIG. 13B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of IL-1a in pg/mL.

FIG. 14 shows hybrid cytokine receptors induce functional activation of T cells in response to ligand stimulation. T cells were transduced with hybrid cytokine receptor SEQ72 (IL4R ECD+IL9R TM+IL9R ICD) and CAR (4D5) and cocultured with its respective ligand (i) before, (ii) during, or (iii) before+during, co-culture with target cells for 160 hrs. Cell culture supernatants were collected and the concentrations of effector cytokines measured by Luminex assay. The graphs shows concentration of IL-2 in pg/mL.

FIGS. 15A-15B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 15A) and SEQ72 (IL4R ECD+IL9R TM+IL9R ICD) and CAR (4D5) (FIG. 15B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of IL-3 in pg/mL.

FIGS. 16A-16B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 16A) and SEQ72 (IL4R ECD+IL9R TM+IL9R ICD) and CAR (4D5) (FIG. 16B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of IL-6 in pg/mL.

FIGS. 17A-17B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 17A) and SEQ72 (IL4R ECD+IL9R TM+IL9R ICD) and CAR (4D5) (FIG. 17B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of IP-10 in pg/mL.

FIGS. 18A-18B show hybrid cytokine receptors induce functional activation of T cells in response to ligand stimulation. T cells were transduced with hybrid cytokine receptors SEQ63 (IL21R ECD+IL9R TM+IL9R ICD) and CAR (4D5) (FIG. 18A) and SEQ72 (IL4R ECD+IL9R TM+IL9R ICD) and CAR (4D5) (FIG. 18B) and cocultured with their respective ligands (i) before, (ii) during, or (iii) before+during, co-culture with target cells for 160 hrs. Cell culture supernatants were collected and the concentrations of effector cytokines measured by Luminex assay. The graphs shows concentration of MIP-1a in pg/mL.

FIG. 19 shows hybrid cytokine receptors induce functional activation of T cells in response to ligand stimulation. T cells were transduced with hybrid cytokine receptor SEQ63 (IL21R ECD+IL9R TM+IL9R ICD) and CAR (4D5) and cocultured with its respective ligand (i) before, (ii) during, or (iii) before+during, co-culture with target cells for 160 hrs. Cell culture supernatants were collected and the concentrations of effector cytokines measured by Luminex assay. The graphs shows concentration of RANTES in pg/mL.

### DETAILED DESCRIPTION OF THE DISCLOSURE

The present disclosure generally relates to, among other things, chimeric switch receptors containing an endodomain of an IL-9 receptor and a binding domain of an endogenous receptor, wherein the chimeric switch receptor is engineered to modulate transcriptional regulation in a ligand-dependent manner. The activity of these switch receptors can be controlled by the presence of an extracellular ligand, allowing for spatial and temporal control of specific gene expression in mammalian cells, as well as for use in modulating cell activities, immune system responses, or in treating various health conditions, such as diseases. Particularly, the chimeric switch receptor (termed “IL-9 switch receptor”), even though containing the endodomain of an IL-9 receptor, does not require IL-9 for activation and can be tailored to be activated by ligands specific for the binding domains of the endogenous receptors, such as normally inhibitory ligands. This class of chimeric switch receptors is synthetic and recombinant, and does not occur in nature. As described below, the chimeric switch receptors disclosed herein can be synthetic polypeptides, and can be engineered, designed, or modified so as to provide desired and/or improved properties, e.g., modulating transcription. The disclosure also provides compositions and methods useful for producing such receptors, nucleic acids encoding same, cells genetically modified with the nucleic acids, as well as methods for modulating an activity of a cell, modulating immune system, and/or for the treatment of various diseases.

In the following detailed description, the illustrative alternatives described in the detailed description and claims are not meant to be limiting. Other alternatives may be used and other changes may be made without departing from the spirit or scope of the subject matter presented here. It will be readily understood that the aspects, as generally described herein, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation as disclosed herein by an administration route including, but not limited to, intravenous, intra-arterial, intracranial, intramuscular, intraperitoneal, subcutaneous, intramuscular, or combinations thereof. The term includes, but is not limited to, administration by a medical professional and self-administration.

“Cancer” refers to the presence of cells possessing several characteristics typical of cancer-causing cells, such as uncontrolled proliferation, immortality, metastatic potential, rapid growth and proliferation rate, and certain characteristic morphological features. Cancer cells can aggregate into a mass, such as a tumor, or can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The terms “cell”, “cell culture”, and “cell line” refer not only to the particular subject cell or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutations (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

As used herein “endogenous” refers to any material from or produced inside an organism, cell, tissue or system (e.g., as it would occur in nature or naturally produced).

The term “percent identity”, as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, and so forth. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### IL-9 And Common Gamma Chain Receptors

Interleukin-9 (IL-9) is a member of a group of cytokines referred to as the common γ chain cytokines. Common y chain cytokines exert numerous functions on immune cell survival, function and proliferation. The γc family consists of six members—IL-2, IL-4, IL-7, IL-9, IL-15, and IL-21—which all have unique receptors. Upon receptor ligation, γc cytokines through JAK1 and JAK3 activate various developmental pathways including STAT1, STAT3, STAT5, MAPK, and PI3K/AKT pathways.

An important aspect of γc receptor signaling is positive and negative regulation of pathways to either enhance or repress signaling. Some of the γc cytokines can mediate similar signaling pathways and transcriptional programs (Kovanen P E, Rosenwald A, Fu J, Hurt E M, Lam L T, Giltnane J M, et al. Analysis of Gamma C-Family Cytokine Target Genes. Identification of Dual-Specificity Phosphatase 5 (DUSP5) as a Regulator of Mitogen-Activated Protein Kinase Activity in Interleukin-2 Signaling. J Biol Chem (2003) 278 (7): 5205-13; Osinalde N, Sanchez-Quiles V, Akimov V, Guerra B, Blagoev B, Kratchmarova I. Simultaneous Dissection and Comparison of IL-2 and IL-15 Signaling Pathways by Global Quantitative Phosphoproteomics. Proteomics (2015) 15 (2-3): 520-31), however, physiological differences in cytokine signaling are mediated by competition for γc between the different receptors (Gonnord P, Angermann B R, Sadtler K, Gombos E, Chappert P, Meier-Schellersheim M, et al. A Hierarchy of Affinities Between Cytokine Receptors and the Common Gamma Chain Leads to Pathway Cross-Talk. Sci Signal (2018) 11 (524)), variability in receptor expression on T cell subsets, a bias for signaling through different STAT molecules, and differences in activation of the MAPK and PI3K pathways (Zeng R, Spolski R, Casas E, Zhu W, Levy D E, Leonard W J. The Molecular Basis of IL-21-Mediated Proliferation. Blood (2007) 109 (10): 4135-42; Gadina M, Sudarshan C, Visconti R, Zhou Y J, Gu H, Neel B G, et al. The Docking Molecule Gab2 is Induced by Lymphocyte Activation and is Involved in Signaling by Interleukin-2 and Interleukin-15 But Not Other Common Gamma Chain-Using Cytokines. J Biol Chem (2000) 275 (35): 26959-66).

The IL-9 receptor alpha (IL-9Rα), a member of the type I hematopoietin receptor superfamily, has high affinity (Kd of approximately 100 pM) for IL-9. This 64-kDa glycoprotein is reported on a variety of hematopoietic cells, particularly T cells. Similar to the other members of the IL-2 receptor family, IL-9Rα also forms a heterotypic receptor complex with the common gamma (γc) chain. In the IL-9R heterocomplex, the IL-9Rα chain is the ligand binding domain and y chain serves as the signaling subunit. The IL-9Rα subunit is characterized by four extracellular cysteines and the conserved WSXWS motif, while the intracellular domain contains a BOX1 consensus sequence and a serine rich region. IL-9Rα is found in both membrane bound and soluble forms, whereas the yc subunit is observed only in a membrane bound form.

IL-9 binding to IL-9Rα results in the formation the IL-9R heterocomplex. A hallmark of the IL-9R heterocomplex is the absence of any intracellular enzymatic activity, and, therefore, Janus kinases (JAK) need to mediate the phosphorylation of the receptor (Knoops L., Renauld J. C. IL-9 and its receptor: From signal transduction to tumorigenesis. Growth Factors. 2004;22:207-215). Upon IL-9 binding to the receptor, a conformational change occurs in the IL-9R heterocomplex, which allows JAK molecules to bind to the proline rich BOX1 motif in the membrane-proximal region of IL-9Rα. JAK1 associates with IL-9Ra, whereas JAK3 binds to γc. Phosphorylated JAK1 and JAK3 then mediate the phosphorylation of receptor tyrosine residues. Phosphorylated tyrosine residues act as docking sites for the downstream Src homology 2 (SH2) domain containing signaling molecules such as Signal Transducer and Activator of Transcription (STAT) transcription factors, insulin receptor substrate (IRS), and the adaptors of the Mitogen-Activated Protein Kinase (MAPK) pathways.

### Compositions of the Disclosure

As described in greater detail below, one aspect of the present disclosure relates to recombinant nucleic acids encoding chimeric switch receptors that include an extracellular portion comprising a binding domain of an endogenous cytokine receptor or endogenous inhibitory receptor, an intracellular portion comprising an endodomain of an IL-9 receptor, and transmembrane domain that joins the extracellular portion and the intracellular portion. Such receptors are engineered to modulate transcriptional regulation in a ligand-dependent manner with various advantages including the ability to convert an otherwise negative signal into a positive signal in the cell. Thus, the present disclosure also encompasses switch receptors that are able to switch negative signals to positive signals for enhancement of an immune response. The present disclosure also encompasses receptors that are able to bind to an endogenous or exogenously given ligand (e.g. a cytokine) and, regardless of the cytokine, result in activation of STAT5, for example, via the common gamma chain and IL-9 endodomain.

As described in the Examples, certain recombinant nucleic acids encoding chimeric receptors can be tested and validated in T cells. These chimeric receptors are expected to show similar performance in mouse models as well as models in other suitable animals or in vitro systems. The receptors disclosed herein may be engineered into various immune cell types for enhanced discrimination and elimination of tumors, or in recombinant host cells for control of autoimmunity and infection. Accordingly, recombinant host cells and compositions of cells, such as immune cells capable of expressing one of more of the chimeric receptors disclosed herein, are also within the scope of the disclosure. In some embodiments, a composition of cells expresses the chimeric receptor encoded by the recombinant nucleic acid described herein.

### Switch Receptors

The present disclosure is based, inter alia, on recombinant nucleic acid molecules encoding chimeric receptors which comprise an endodomain of an IL-9 receptor, thus creating chimeric receptors that can respond to various extracellular ligands while maintaining the ability to initiate intracellular signaling through the IL-9 receptor endodomain. Immune cells expressing these chimeric receptors may be useful in the context of modulating immune cell activity. In some embodiments, the ligand can be added exogenously and not be limited to production within the cell.

As outlined above, some embodiments of the present disclosure relate to recombinant nucleic acid molecules encoding chimeric receptors containing the endodomain of an IL-9 receptor. In particular, the chimeric receptors, even though containing an IL-9 endodomain, do not require binding of IL-9 for the functioning of the receptors. Generally, the chimeric receptors comprise an extracellular portion, an intracellular portion comprising an endodomain of an IL-9 receptor, and a transmembrane domain that joins the extracellular portion and the intracellular portion. In some embodiments, extracellular portion comprises a binding domain of an endogenous cytokine receptor. In some embodiments, the extracellular portion comprises a binding domain of an endogenous inhibitory receptor.

In some embodiments, provided herein is a recombinant nucleic acid encoding a chimeric polypeptide including: (a) an extracellular portion comprising a binding domain of an endogenous cytokine receptor; (b) an intracellular portion comprising an endodomain of an IL-9 receptor; (c) a transmembrane domain that joins the extracellular portion and the intracellular portion.

In some embodiments, provided herein is a recombinant nucleic acid encoding a chimeric polypeptide including: (a) an extracellular portion comprising a binding domain of an endogenous inhibitory receptor; (b) an intracellular portion comprising an endodomain of an IL-9 receptor linked to a BOX½ common gamma chain domain; (c) a transmembrane domain that joins the extracellular portion and the intracellular portion.

In some embodiments, provided herein is a recombinant nucleic acid encoding a

chimeric polypeptide including: (a) an extracellular portion comprising a binding domain of an endogenous inhibitory receptor linked to an agent specific for the common gamma chain; (b) an intracellular portion comprising an endodomain of an IL-9 receptor; (c) a transmembrane domain that joins the extracellular portion and the intracellular portion.

### Extracellular Portions

As outlined above, the extracellular portions of the chimeric receptors (e.g., switch receptors) in some embodiments of the disclosure have a binding domain of an endogenous cytokine receptor or an endogenous inhibitory receptor. A binding domain of an endogenous cytokine receptor can be an extracellular portion of an endogenous cytokine receptor, or a fragment or truncation thereof that can bind a cytokine polypeptide sequence. In some embodiments, the endogenous cytokine receptor is a member of the common gamma chain receptor family. Members of the common gamma chain receptor family are known in the art and are discussed supra. In some embodiments, the endogenous cytokine receptor is selected from IL-2rb, IL-2ra, IL-4r, IL-7ra, IL-15ra, and IL-21ra. As described supra, IL-2rb, IL-2ra, IL-4r, IL-7ra, IL-15ra, and IL-21ra are all part of the common gamma chain family. Therefore, these receptors are capable of recruiting the common gamma chain, upon ligand binding, and signaling can proceed through the IL-9 endodomain.

In one embodiment, the endogenous cytokine receptor comprises the amino acid sequence of IL-2rb (SEQ ID NO:1) below:

AVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFE
NLRLMAPISLQVVHVETHRCNISWEISQASHYFERHLEFEARTLSPGHTWE
EAPLLTLKQKQEWICLETLTPDTQYEFQVRVKPLQGEFTTWSPWSQPLAFR
TKPAALGKDT

In one embodiment, the endogenous cytokine receptor comprises the amino acid sequence of IL-2ra (SEQ ID NO:2) below:

ELCDDDPPEIPHATFKAMAYKEGTMLNCECKRGFRRIKSGSLYMLCTGNSS
HSSWDNQCQCTSSATRNTTKQVTPQPEEQKERKTTEMQSPMQPVDQASLPG
HCREPPPWENEATERIYHFVVGQMVYYQCVQGYRALHRGPAESVCKMTHGK
TRWTQPQLICTGEMETSQFPGEEKPQASPEGRPESETSCLVTTTDFQIQTE
MAATMETSIFTTEYQ

In one embodiment, the endogenous cytokine receptor comprises the amino acid sequence of IL-4 (SEQ ID NO:3) below:

MKVLQEPTCVSDYMSISTCEWKMNGPTNCSTELRLLYQLVFLLSEAHTCIP
ENNGGAGCVCHLLMDDVVSADNYTLDLWAGQQLLWKGSFKPSEHVKPRAPG
NLTVHTNVSDTLLLTWSNPYPPDNYLYNHLTYAVNIWSENDPADFRIYNVT
YLEPSLRIAASTLKSGISYRARVRAWAQCYNTTWSEWSPSTKWHNSYREPF
EQH

In one embodiment, the endogenous cytokine receptor comprises the amino acid sequence of IL-7ra (SEQ ID NO:4) below:

ESGYAQNGDLEDAELDDYSFSCYSQLEVNGSQHSLTCAFEDPDVNITNLEF
EICGALVEVKCLNFRKLQEIYFIETKKFLLIGKSNICVKVGEKSLTCKKID
LTTIVKPEAPFDLSVVYREGANDFVVTFNTSHLQKKYVKVLMHDVAYRQEK
DENKWTHVNLSSTKLTLLQRKLQPAAMYEIKVRSIPDHYFKGFWSEWSPSY
YFRTPEINNSSGEMD

In one embodiment, the endogenous cytokine receptor comprises the amino acid sequence of IL-15ra (SEQ ID NO:5) below:

ITCPPPMSVEHADIWVKSYSLYSRERYICNSGFKRKAGTSSLTECVLNKAT
NVAHWTTPSLKCIRDPALVHQRPAPPSTVTTAGVTPQPESLSPSGKEPAAS
SPSSNNTAATTAAIVPGSQLMPSKSPSTGTTEISSHESSHGTPSQTTAKNW
ELTASASHQPPGVYPQGHSDTT

In one embodiment, the endogenous cytokine receptor comprises the amino acid sequence of IL-21ra (SEQ ID NO:6) below:

CPDLVCYTDYLQTVICILEMWNLHPSTLTLTWQDQYEELKDEATSCSLHRS
AHNATHATYTCHMDVFHFMADDIFSVNITDQSGNYSQECGSFLLAESIKPA
PPFNVTVTFSGQYNISWRSDYEDPAFYMLKGKLQYELQYRNRGDPWAVSPR
RKLISVDSRSVSLLPLEFRKDSSYELQVRAGPMPGSSYQGTWSEWSDPVIF
QTQSEELKE

In some embodiments, the extracellular portions of the chimeric polypeptides disclosed herein (e.g., IL-9 switch receptors) having at least 80% sequence identity, such as, at least 80%, at least 85%, at least 90%, at least 95%, at least 96%, at least 97%, at least 98%, or 99% sequence identity to a sequence selected from the group consisting of SEQ ID NOS: 1-6 in the Sequence Listing. In some embodiments, the extracellular portion includes an amino acid sequence having at least 90% sequence identity to a sequence selected from the group consisting of SEQ ID NOS: 1-6. In some embodiments, the extracellular portion includes an amino acid sequence having at least 95% sequence identity to a sequence selected from the group consisting of SEQ ID NOS: 1-6. In some embodiments, the extracellular portion includes an amino acid sequence having about 100% sequence identity to a sequence selected from the group consisting of SEQ ID NOS: 1-6. In some embodiments, the extracellular portion includes an amino acid sequence having a sequence selected from the group consisting of SEQ ID NOS: 1-6, wherein one, two, three, four, or five of the amino acid residues in any one of the SEQ ID NOS: 1-6 is substituted by a different amino acid residue.

In another aspect of the present disclosure, the extracellular portion of the chimeric receptor of the disclosure can also comprise a binding domain of an endogenous inhibitory receptor. Specifically, as described supra, in some embodiments, provided herein is a recombinant nucleic acid encoding a chimeric polypeptide including: (a) an extracellular portion comprising a binding domain of an endogenous inhibitory receptor linked to an agent specific for the common gamma chain; (b) an intracellular portion comprising an endodomain of an IL-9 receptor; (c) a transmembrane domain that joins the extracellular portion and the intracellular portion.

As described supra, signaling through the IL-9 endodomain requires recruitment of the common gamma chain. In some embodiments, the binding domains of the extracellular portion of the inhibitory receptors described herein are not able to naturally recruit the common gamma chain in order to elicit signaling through the IL-9 endodomain of the chimeric receptor. Accordingly, in these embodiments, the binding domain of the endogenous inhibitory receptor is linked to an agent specific for the common gamma chain. The binding domain of the inhibitory receptor is linked to the agent specific for the common gamma chain such that both components are able to function in their intended way (e.g., the binding domain is able to bind a ligand and the agent specific for the common gamma chain is able to bind the common gamma chain).

In some embodiments, the agent specific for the common gamma chain comprises agent specific for the common gamma chain comprises a nanobody, a darpin, IL-2, IL-4, IL-7, or an scFv.

In some embodiments, an scFv directed to the common gamma chain is cloned in frame with the extracellular portion of the chimeric receptor, with suitable linker sequences inserted between these components. The binding of the common gamma chain to the scFv will be sufficient to induce chimeric receptor dimerization.

scFVs directed to the common gamma chain as well as their sequences are known in the art and described in WO 2017/021540. However, the use of scFVs directed to the common gamma chain with respect to switch receptors is not disclosed therein.

A binding domain of an endogenous inhibitory receptor can be an extracellular portion of an endogenous inhibitory receptor, or a fragment or truncation thereof that can bind a cytokine polypeptide sequence and subsequently decrease immune activity. For example, a natural inhibitory receptor can reduce T cell proliferation, T cell survival, cytokine release, or immune cell lytic activity upon binding of a natural agonist.

Endogenous inhibitory receptors are well known in the art and are contemplated for use in the compositions described herein (Turnis et al., “Inhibitory Receptors as Targets for Cancer Immunotherapy,” Eur J Immunol 2015 45 (7): 1892-1905).

In some embodiments, the endogenous inhibitory receptor signals through trimerization. In some embodiments, the endogenous inhibitory receptor is a member of the TNF receptor superfamily.

In some embodiments, the endogenous inhibitory receptor signals as functional dimers of dimers. In some embodiments, the endogenous inhibitory receptor is a member of the TGF beta superfamily of receptors. Receptors in this family include, for example, Type I, Type II, and Type III receptors. Exemplary members of the Type I receptor family include, without limitation, ACVRL1, ACVR1A, BMPR1A, ACVR1B, TGFβR1, BMPR1B, and ACVR1C. Exemplary members of the Type II receptor family include, without limitation, TGFBR2, BMPR2, ACVR2A, ACVR2B, and AMHR2. TGF βR3 is a member of the Type III family of receptors.

In some embodiments, the endogenous inhibitory receptor signal as dimers. In some embodiments, the endogenous inhibitory receptor is a member of the VEGF family of receptors. Receptors in this family include, for example, VEGFR1, VEGFR2, and VEGFR3.

In some embodiments, the endogenous inhibitory receptor is selected from TGF-beta R1, TGF-beta R2, IL-10ra, FAS, CTLA4, LAG3, TIM3, PD1, ILT2, ILT3, ILT4, ILT5, and VEGF. Thus, in some embodiments, the extracellular portion of the chimeric receptor of the disclosure can be a binding domain of TGF-beta R1, TGF-beta R2, IL-10ra, FAS, CTLA4, LAG3, TIM3, PD1, ILT2, ILT3, ILT4, ILT5, and VEGF.

In one embodiment, the endogenous inhibitory receptor comprises the amino acid sequence of TGFBR1 (SEQ ID NO:7) below:

LQCFCHLCTKDNFTCVTDGLCFVSVTETTDKVIHNSMCIAEIDLIPRDRPF
VCAPSSKTGSVTTTYCCNQDHCNKIELPTTVKSSPGLGPVEL

In one embodiment, the endogenous inhibitory receptor comprises the amino acid sequence of TGFBR2 (SEQ ID NO:8) below:

TIPPHVQKSVNNDMTVTDNNGAVKFPQLCKFCDVRFSTCDNQKSCMSNCSI
TSICEKPQEVCVAVWRKNDENITLETVCHDPKLPYHDFILEDAASPKCIMK
EKKKPGETFFMCSCSSDECNDNIIFSEEYNTSNPDLLLVIFQ

In one embodiment, the endogenous inhibitory receptor comprises the amino acid sequence of IL-10ra (SEQ ID NO:9) below:

HGTELPSPPSVWFEAEFFHHILHWTPIPNQSESTCYEVALLRYGIESWNSI
SNCSQTLSYDLTAVTLDLYHSNGYRARVRAVDGSRHSNWTVTNTRFSVDEV
TLTVGSVNLEIHNGFILGKIQLPRPKMAPANDTYESIFSHFREYEIAIRKV
PGNFTFTHKKVKHENFSLLTSGEVGEFCVQVKPSVASRSNKGMWSKEECIS
LTRQYFTVTN

In one embodiment, the endogenous inhibitory receptor comprises the amino acid sequence of FAS (SEQ ID NO:10) below:

QVTDINSKGLELRKTVTTVETQNLEGLHHDGQFCHKPCPPGERKARDCTVN
GDEPDCVPCQEGKEYTDKAHFSSKCRRCRLCDEGHGLEVEINCTRTQNTKC
RCKPNFFCNSTVCEHCDPCTKCEHGIIKECTLTSNTKCKEEGSRSN

In one embodiment, the endogenous inhibitory receptor comprises the amino acid sequence of CTLA4 (SEQ ID NO:11) below:

KAMHVAQPAVVLASSRGIASFVCEYASPGKATEVRVTVLRQADSQVTEVCA
ATYMMGNELTFLDDSICTGTSSGNQVNLTIQGLRAMDTGLYICKVELMYPP
PYYLGIGNGTQIYVIDPEPCPDSD

In one embodiment, the endogenous inhibitory receptor comprises the amino acid sequence of LAG3 (SEQ ID NO:12) below:

LQPGAEVPVVWAQEGAPAQLPCSPTIPLQDLSLLRRAGVTWQHQPDSGPPA
AAPGHPLAPGPHPAAPSSWGPRPRRYTVLSVGPGGLRSGRLPLQPRVQLDE
RGRQRGDFSLWLRPARRADAGEYRAAVHLRDRALSCRLRLRLGQASMTASP
PGSLRASDWVILNCSFSRPDRPASVHWFRNRGQGRVPVRESPHHHLAESFL
FLPQVSPMDSGPWGCILTYRDGFNVSIMYNLTVLGLEPPTPLTVYAGAGSR
VGLPCRLPAGVGTRSFLTAKWTPPGGGPDLLVTGDNGDFTLRLEDVSQAQA
GTYTCHIHLQEQQLNATVTLAIITVTPKSFGSPGSLGKLLCEVTPVSGQER
FVWSSLDTPSQRSFSGPWLEAQEAQLLSQPWQCQLYQGERLLGAAVYFTEL
SSPGAQRSGRAPGALPAGHL

In one embodiment, the endogenous inhibitory receptor comprises the amino acid sequence of TIM3 (SEQ ID NO:13) below:

SEVEYRAEVGQNAYLPCFYTPAAPGNLVPVCWGKGACPVFECGNVVLRTDE
RDVNYWTSRYWLNGDFRKGDVSLTIENVTLADSGIYCCRIQIPGIMNDEKF
NLKLVIKPAKVTPAPTRQRDFTAAFPRMLTTRGHGPAETQTLGSLPDINLT
QISTLANELRDSRLANDLRDSGATIRIG

In one embodiment, the endogenous inhibitory receptor comprises the amino acid sequence of PD1 (SEQ ID NO:14) below:

FTVTVPKDLYVVEYGSNMTIECKFPVEKQLDLAALIVYWEMEDKNHIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNER

In one embodiment, the endogenous inhibitory receptor comprises the amino acid sequence of ILT2 (SEQ ID NO:15) below:

GHLPKPTLWAEPGSVITQGSPVTLRCQGGQETQEYRLYREKKTALWITRI
PQELVKKGQFPIPSITWEHAGRYRCYYGSDTAGRSESSDPLELVVTGAYI
KPTLSAQPSPVVNSGGNVILQCDSQVAFDGFSLCKEGEDEHPQCLNSQPH
ARGSSRAIFSVGPVSPSRRWWYRCYAYDSNSPYEWSLPSDLLELLVLGVS
KKPSLSVQPGPIVAPEETLTLQCGSDAGYNRFVLYKDGERDFLQLAGAQP
QAGLSQANFTLGPVSRSYGGQYRCYGAHNLSSEWSAPSDPLDILIAGQFY
DRVSLSVQPGPTVASGENVTLLCQSQGWMQTFLLTKEGAADDPWRLRSTY
QSQKYQAEFPMGPVTSAHAGTYRCYGSQSSKPYLLTHPSDPLELVVSGPS
GGPSSPTTGPTSTSGPEDQPLTPTGSDPQSGLGRHLGV

In one embodiment, the endogenous inhibitory receptor comprises the amino acid sequence of ILT3 (SEQ ID NO:16) below:

QAGPLPKPTLWAEPGSVISWGNSVTIWCQGTLEAREYRLDKEESPAPWDR
QNPLEPKNKARFSIPSMTEDYAGRYRCYYRSPVGWSQPSDPLELVMTGAY
SKPTLSALPSPLVTSGKSVTLLCQSRSPMDTFLLIKERAAHPLLHLRSEH
GAQQHQAEFPMSPVTSVHGGTYRCFSSHGFSHYLLSHPSDPLELIVSGSL
EDPRPSPTRSVSTAAGPEDQPLMPTGSVPHSGLRRHWE

In one embodiment, the endogenous inhibitory receptor comprises the amino acid sequence of ILT4 (SEQ ID NO:17) below:

QTGTIPKPTLWAEPDSVITQGSPVTLSCQGSLEAQEYRLYREKKSASWIT
RIRPELVKNGQFHIPSITWEHTGRYGCQYYSRARWSELSDPLVLVMTGAY
PKPTLSAQPSPVVTSGGRVTLQCESQVAFGGFILCKEGEEEHPQCLNSQP
HARGSSRAIFSVGPVSPNRRWSHRCYGYDLNSPYVWSSPSDLLELLVPGV
SKKPSLSVQPGPVVAPGESLTLQCVSDVGYDRFVLYKEGERDLRQLPGRQ
PQAGLSQANFTLGPVSRSYGGQYRCYGAHNLSSECSAPSDPLDILITGQI
RGTPFISVQPGPTVASGENVTLLCQSWRQFHTFLLTKAGAADAPLRLRSI
HEYPKYQAEFPMSPVTSAHAGTYRCYGSLNSDPYLLSHPSEPLELVVSGP
SMGSSPPPTGPISTPAGPEDQPLTPTGSDPQSGLGRHLGV

In one embodiment, the endogenous inhibitory receptor comprises the amino acid sequence of ILT5 (SEQ ID NO:18) below:

GPFPKPTLWAEPGSVISWGSPVTIWCQGSQEAQEYRLHKEGSPEPLDRNN
PLEPKNKARFSIPSMTEHHAGRYRCHYYSSAGWSEPSDPLEMVMTGAYSK
PTLSALPSPVVASGGNMTLRCGSQKGYHHFVLMKEGEHQLPRTLDSQQLH
SRGFQALFPVGPVTPSHRWRFTCYYYYTNTPWVWSHPSDPLEILPSGVSR
KPSLLTLQGPVLAPGQSLTLQCGSDVGYNRFVLYKEGERDFLQRPGQQPQ
AGLSQANFTLGPVSPSNGGQYRCYGAHNLSSEWSAPSDPLNILMAGQIYD
TVSLSAQPGPTVASGENVTLLCQSWWQFDTFLLTKEGAAHPPLRLRSMYG
AHKYQAEFPMSPVTSAHAGTYRCYGSYSSNPHLLSHPSEPLELVVSGHSG
GSSLPPTGPPSTPGLGRYLE

In one embodiment, the endogenous inhibitory receptor comprises the amino acid sequence of VEGFR1 (SEQ ID NO:19) below:

SKLKDPELSLKGTQHIMQAGQTLHLQCRGEAAHKWSLPEMVSKESERLSI
TKSACGRNGKQFCSTLTLNTAQANHTGFYSCKYLAVPTSKKKETESAIYI
FISDTGRPFVEMYSEIPEIIHMTEGRELVIPCRVTSPNITVTLKKFPLDT
LIPDGKRIIWDSRKGFIISNATYKEIGLLTCEATVNGHLYKTNYLTHRQT
NTIIDVQISTPRPVKLLRGHTLVLNCTATTPLNTRVQMTWSYPDEKNKRA
SVRRRIDQSNSHANIFYSVLTIDKMQNKDKGLYTCRVRSGPSFKSVNTSV
HIYDKAFITVKHRKQQVLETVAGKRSYRLSMKVKAFPSPEVVWLKDGLPA
TEKSARYLTRGYSLIIKDVTEEDAGNYTILLSIKQSNVFKNLTATLIVNV
KPQIYEKAVSSFPDPALYPLGSRQILTCTAYGIPQPTIKWFWHPCNHNHS
EARCDFCSNNEESFILDADSNMGNRIESITQRMAIIEGKNKMASTLVVAD
SRISGIYICIASNKVGTVGRNISFYITDVPNGFHVNLEKMPTEGEDLKLS
CTVNKFLYRDVTWILLRTVNNRTMHYSISKQKMAITKEHSITLNLTIMNV
SLQDSGTYACRARNVYTGEEILQKKEITIRDQEAPYLLRNLSDHTVAISS
STTLDCHANGVPEPQITWFKNNHKIQQEPGIILGPGSSTLFIERVTEEDE
GVYHCKATNQKGSVESSAYLTVQGTSDKSNLE

In one embodiment, the endogenous inhibitory receptor comprises the amino acid sequence of VEGFR2 (SEQ ID NO:20) below:

ASVGLPSVSLDLPRLSIQKDILTIKANTTLQITCRGQRDLDWLWPNNQSG
SEQRVEVTECSDGLFCKTLTIPKVIGNDTGAYKCFYRETDLASVIYVYVQ
DYRSPFIASVSDQHGVVYITENKNKTVVIPCLGSISNLNVSLCARYPEKR
FVPDGNRISWDSKKGFTIPSYMISYAGMVFCEAKINDESYQSIMYIVVVV
GYRIYDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKNS
TFVRVHEKPFVAFGSGMESLVEATVGERVRIPAKYLGYPPPEIKWYKNGI
PLESNHTIKAGHVLTIMEVSERDTGNYTVILTNPISKEKQSHVVSLVVYV
PPQIGEKSLISPVDSYQYGTTQTLTCTVYAIPPPHHIHWYWQLEEECANE
PSQAVSVTNPYPCEEWRSVEDFQGGNKIEVNKNQFALIEGKNKTVSTLVI
QAANVSALYKCEAVNKVGRGERVISFHVTRGPEITLQPDMQPTEQESVSL
WCTADRSTFENLTWYKLGPQPLPIHVGELPTPVCKNLDTLWKLNATMFSN
STNDILIMELKNASLQDQGDYVCLAQDRKTKKRHCVVRQLTVLERVAPTI
TGNLENQTTSIGESIEVSCTASGNPPPQIMWFKDNETLVEDSGIVLKDGN
RNLTIRRVRKEDEGLYTCQACSVLGCAKVEAFFIIEGAQEKTNLE

In one embodiment, the endogenous inhibitory receptor comprises the amino acid sequence of VEGFR3 (SEQ ID NO:21) below:

YSMTPPTLNITEESHVIDTGDSLSISCRGQHPLEWAWPGAQEAPATGDKD
SEDTGVVRDCEGTDARPYCKVLLLHEVHANDTGSYVCYYKYIKARIEGTT
AASSYVFVRDFEQPFINKPDTLLVNRKDAMWVPCLVSIPGLNVTLRSQSS
VLWPDGQEVVWDDRRGMLVSTPLLHDALYLQCETTWGDQDFLSNPFLVHI
TGNELYDIQLLPRKSLELLVGEKLVLNCTVWAEFNSGVTFDWDYPGKQAE
RGKWVPERRSQQTHTELSSILTIHNVSQHDLGSYVCKANNGIQRFRESTE
VIVHENPFISVEWLKGPILEATAGDELVKLPVKLAAYPPPEFQWYKDGKA
LSGRHSPHALVLKEVTEASTGTYTLALWNSAAGLRRNISLELVVNVPPQI
HEKEASSPSIYSRHSRQALTCTAYGVPLPLSIQWHWRPWTPCKMFAQRSL
RRRQQQDLMPQCRDWRAVTTQDAVNPIESLDTWTEFVEGKNKTVSKLVIQ
NANVSAMYKCVVSNKVGQDERLIYFYVTTIPDGFTIESKPSEELLEGQPV
LLSCQADSYKYEHLRWYRLNLSTLHDAHGNPLLLDCKNVHLFATPLAASL
EEVAPGARHATLSLSIPRVAPEHEGHYVCEVQDRRSHDKHCHKKYLSVQA
LEAPRLTQNLTDLLVNVSDSLEMQCLVAGAHAPSIVWYKDERLLEEKSGV
DLADSNQKLSIQRVREEDAGRYLCSVCNAKGCVNSSASVAVEGSEDKGSM
E

A number of other receptor sequences are contemplated for use in the extracellular portions of the chimeric receptors described herein. Such sequences may be used to switch the natural biology of the receptor ligand or provide for localized stimulation of a cell population. Exemplary sequences of the extracellular portions of such receptors are shown in Table 1 below.

| Receptor | | |
|---|---|---|
| Extracellular | | SEQ ID |
| Portion | Amino Acid Sequence | NO |
| OPG | ETFPPKYLHYDEETSHQLLCDKCPPGTYLKQHCTAKWKTVCAPCPDHYYTDSWHT | 22 |
| | SDECLYCSPVCKELQYVKQECNRTHNRVCECKEGRYLEIEFCLKHRSCPPGFGVVQ | |
| | AGTPERNTV | |
| | CKRCPDGFFSNETSSKAPCRKHTNCSVFGLLLTQKGNATHDNICSGNSESTQKCGI | |
| | DVTLCEEAFFRFAVPTKFTPNWLSVLVDNLPGTKVNAESVERIKRQHSSQEQTFQL | |
| | LKLWKHQN | |
| | KDQDIVKKIIQDIDLCENSVQRHIGHANLTFEQLRSLMESLPGKKVGAEDIEKTIKAC | |
| | KPSDQILKLLSLWRIKNGDQDTLKGLMHALKHSKTYHFPKTVTQSLKKTIRFLHSFT | |
| | MYKLYQKLFLEMIGNQVQSVKISCL | |
| TACI | MSGLGRSRRGGRSRVDQEERFPQGLWTGVAMRSCPEEQYWDPLLGTCMSCKTI | 23 |
| | CNHQSQRTCAAFCRSLSCRKEQGKFYDHLLRDCISCASICGQHPKQCAYFCENKLR | |
| | SPVNLPPELRRQRSGEVENNSDNSGRYQGLEHRGSEASPALPGLKLSADQVALVYS | |
| BCMA | MLQMAGQCSQNEYFDSLLHACIPCQLRCSSNTPPLTCQRYCNASVTNSVKGTNA | 24 |
| NGFR | KEACPTGLYTHSGECCKACNLGEGVAQPCGANQTVCEPCLDSVTFSDVVSATEPC | 25 |
| | KPCTECVGLQSMSAPCVEADDAVCRCAYGYYQDETTGRCEACRVCEAGSGLVFSC | |
| | QDKQNTVCEE | |
| | CPDGTYSDEANHVDPCLPCTVCEDTERQLRECTRWADAECEEIPGRWITRSTPPE | |
| | GSDSTAPSTQEPEAPPEQDLIASTVAGVVTTVMGSSQPVVTRGTTDN | |
| EDAR | EYSNCGENEYYNQTTGLCQECPPCGPGEEPYLSCGYGTKDEDYGCVPCPAEKFSKG | 26 |
| | GYQICRRHKDCEGFFRATVLTPGDMENDAECGPCLPGYYIVILENRPRNIYGIVIVCY | |
| | SCLLAPPNTKECVGATSGASANFPGTSGSSTLSPFQHAHKELSGQGHLATA | |
| DCR2 | ATIPRQDEVPQQTVAPQQQRRSLKEEECPAGSHRSEYTGACNPCTEGVDYTIASN | 27 |
| (TNFRSF10D) | NLPSCLLCTVCKSGQTNKSSCTTTRDTVCQCEKGSFQDKNSPEMCRTCRTGCPRG | |
| | MVKVSNCTPRSDIKCKNESAASSTGKTPAAEETVTTILGMLASPYH | |
| DCR1 | ATTARQEEVPQQTVAPQQQRHSFKGEECPAGSHRSEHTGACNPCTEGVDYTNAS | 28 |
| (TNFRSF10C) | NNEPSCFPCTVCKSDQKHKSSCTMTRDTVCQCKEGTFRNENSPEMCRKCSRCPSG | |
| | EVQVSNCTSWDDIQCVEEFGANATVETPAAEETMNTSPGTPAPAAEETMNTSPG | |
| | TPAPAAEETMTTSPGTPAPAAEETMTTSPGTPAPAAEETMITSPGTPA | |
| CD40 | EPPTACREKQYLINSQCCSLCQPGQKLVSDCTEFTETECLPCGESEFLDTWNRETHC | 29 |
| | HQHKYCDPNLGLRVQQKGTSETDTICTCEEGWHCTSEACESCVLHRSCSPGFGVK | |
| | QIATGVSDTICEPCPVGFFSNVSSAFEKCHPWTSCETKDLVVQQAGTNKTDVVCG | |
| | PQDRLR | |
| DR4 | ASGTEAAAATPSKVWGSSAGRIEPRGGGRGALPTSMGQHGPSARARAGRAPGP | 30 |
| | RPAREASPRLRVHKTFKFVVVGVLLQVVPSSAATIKLHDQSIGTQQWEHSPLGELC | |
| | PPGSHRSEHPGACNRCTEGVGYTNASNNLFACLPCTACKSDEEERSPCTTTRNTAC | |
| | QCKPGTFRNDNSAEMCRKCSRGCPRGMVKVKDCTPWSDIECVHKESGNGHN | |
| DR6 | QPEQKASNLIGTYRHVDRATGQVLTCDKCPAGTYVSEHCTNTSLRVCSSCPVGTFT | 31 |
| | RHENGIEKCHDCSQPCPWPMIEKLPCAALTDRECTCPPGMFQSNATCAPHTVCP | |
| | VGWGVRKKGTETEDVRCKQCARGTFSDVPSSVMKCKAYTDCLSQNLVVIKPGTKE | |
| | TDNVCGTLPSFSSSTSPSPGTAIFPRPEHMETHEVPSSTYVPKGMNSTESNSSASVR | |
| | PKVLSSIQEGTVPDNTSSARGKEDVNKTLPNLQVVNHQQGPHHRHILKLLPSMEA | |
| | TGGEKSSTPIKGPKRGHPRQNLHKHFDINEH | |
| DR5 | ITQQDLAPQQRAAPQQKRSSPSEGLCPPGHHISEDGRDCISCKYGQDYSTHWNDL | 32 |
| | LFCLRCTRCDSGEVELSPCTTTRNTVCQCEEGTFREEDSPEMCRKCRTGCPRGMVK | |
| | VGDCTPWSDIECVHKESGTKHSGEVPAVEETVTSSPGTPASPCS | |
| DR3 | QGGTRSPRCDCAGDFHKKIGLFCCRGCPAGHYLKAPCTEPCGNSTCLVCPQDTFLA | 33 |
| | WENHHNSECARCQACDEQASQVALENCSAVADTRCGCKPGWFVECQVSQCVSS | |
| | SPFYCQPCLDCGALHRHTRLLCSRRDTDCGTCLPGFYEHGDGCVSCPTSTLGSCPE | |
| | RCAAVCGWRQ | |
| TNFRSF1B | LPAQVAFTPYAPEPGSTCRLREYYDQTAQMCCSKCSPGQHAKVFCTKTSDTVCDS | 34 |
| | CEDSTYTQLWNWVPECLSCGSRCSSDQVETQACTREQNRICTCRPGWYCALSKQ | |
| | EGCRLCAPLRKCRPGFGVARPGTETSDVVCKPCAPGTFSNTTSSTDICRPHQICNV | |
| | VAIPGNASMDAVCTSTSPTRSMAPGAVHLPQPVSTRSQHTQPTPEPSTAPSTSFLL | |
| | PMGPSPPAEGSTGD | |
| TNFRSF1 | LVPHLGDREKRDSVCPQGKYIHPQNNSICCTKCHKGTYLYNDCPGPGQDTDCREC | 35 |
| | ESGSFTASENHLRHCLSCSKCRKEMGQVEISSCTVDRDTVCGCRKNQYRHYWSEN | |
| | LFQCFNCSLCLNGTVHLSCQEKQNTVCTCHAGFFLRENECVSCSNCKKSLECTKLCL | |
| | PQIENVKGTEDSGTT | |
| BMPR1B | KKEDGESTAPTPRPKVLRCKCHHHCPEDSVNNICSTDGYCFTMIEEDDSGLPVVTS | 36 |
| | GCLGLEGSDFQCRDTPIPHQRRSIECCTERNECNKDLHPTLPPLKNRDFVDGPIHH | |
| | R | |
| BMPR1A | QNLDSMLHGTGMKSDSDQKKSENGVTLAPEDTLPFLKCYCSGHCPDDAINNTCIT | 37 |
| | NGHCFAIIEEDDQGETTLASGCMKYEGSDFQCKDSPKAQLRRTIECCRTNLCNQYL | |
| | QPTLPPVVIGPFFDGSIR | |
| BMPR2 | SQNQERLCAFKDPYQQDLGIGESRISHENGTILCSKGSTCYGLWEKSKGDINLVKQ | 38 |
| | GCWSHIGDPQECHYEECVVTTTPPSIQNGTYRFCCCSTDLCNVNFTENFPPPDTTP | |
| | LSPPHSFNRDET | |
| CSF3R | ECGHISVSAPIVHLGDPITASCIIKQNCSHLDPEPQILWRLGAELQPGGRQQRLSDG | 39 |
| | TQESIITLPHLNHTQAFLSCCLNWGNSLQILDQVELRAGYPPAIPHNLSCLMNLTTS | |
| | SLICQWEPGPETHLPTSFTLKSFKSRGNCQTQGDSILDCVPKDGQSHCCIPRKHLLL | |
| | YQNMGIWVQAENALGTSMSPQLCLDPMDVVKLEPPMLRTMDPSPEAAPPQAG | |
| | CLQLCWEPWQPGLHINQKCELRHKPQRGEASWALVGPLPLEALQYELCGLLPATA | |
| | YTLQIRCIRWPLPGHWSDWSPSLELRTTERAPTVRLDTWWRQRQLDPRTVQLFW | |
| | KPVPLEEDSGRIQGYVVSWRPSGQAGAILPLCNTTELSCTFHLPSEAQEVALVAYNS | |
| | AGTSRPTPVVFSESRGPALTRLHAMARDPHS | |
| | LWVGWEPPNPWPQGYVIEWGLGPPSASNSNKTWRMEQNGRATGFLLKENIRP | |
| | FQLYEIIVTPLYQDTMGPSQHVYAYSQEMAPSHAPELHLKHIGKTWAQLEWVPEP | |
| | PELGKSPLTHYTIFWTNAQNQSFSAILNASSRGFVLHGLEPASLYHIHLMAASQAG | |
| | ATNSTVLTLMTLTPEGSELH | |
| CSF1R | IPVIEPSVPELVVKPGATVTLRCVGNGSVEWDGPPSPHWTLYSDGSSSILSTNNATF | 40 |
| | QNTGTYRCTEPGDPLGGSAAIHLYVKDPARPWNVLAQEVVVFEDQDALLPCLLTD | |
| | PVLEAGVSLVRVRGRPLMRHTNYSFSPWHGFTIHRAKFIQSQDYQCSALMGGRK | |
| | VMSISIRLKVQKVIPGPPALTLVPAELVRIRGEAAQIVCSASSVDVNFDVFLQHNNT | |
| | KLAIPQQSDFHNNRYQKVLTLNLDQVDFQHAGNYSCVASNVQGKHSTSMFFRVV | |
| | ESAYLNLSSEQNLIQEVTVGEGLNLKVMVEAYPGLQGFNWTYLGPFSDHQPEPKL | |
| | ANATTKDTYRHTFTLSLPRLKPSEAGRYSFLARNPGGWRALTFELTLRYPPEVSVIW | |
| | TFINGSGTLLCAASGYPQPNVTWLQCSGHTDRCDEAQVLQVWDDPYPEVLSQEP | |
| | FHKVTVQSLLTVETLEHNQTYECRAHNSVGSGSWAFIPISAGAHTHPPDEFLFTP | |
| Activin R1A | MEDEKPKVNPKLYMCVCEGLSCGNEDHCEGQQCFSSLSINDGFHVYQKGCFQVY | 41 |
| | EQGKMTCKTPPSPGQAVECCQGDWCNRNITAQLPTKGKSFPGTQNFHLE | |
| Activin R1B | SGPRGVQALLCACTSCLQANYTCETDGACMVSIFNLDGMEHHVRTCIPKVELVPA | 42 |
| | GKPFYCLSSEDLRNTHCCYTDYCNRIDLRVPSGHLKEPEHPSMWGPVE | |
| Activin R1C | LSPGLKCVCLLCDSSNFTCQTEGACWASVMLTNGKEQVIKSCVSLPELNAQVFCHS | 43 |
| | SNNVTKTECCFTDFCNNITLHLPTASPNAPKLGPME | |
| Activin R2B | SGRGEAETRECIYYNANWELERTNQSGLERCEGEQDKRLHCYASWRNSSGTIELVK | 44 |
| | KGCWLDDFNCYDRQECVATEENPQVYFCCCEGNFCNERFTHLPEAGGPEVTYEPP | |
| | PTAPTLLT | |
| Activin R2A | AILGRSETQECLFFNANWEKDRTNQTGVEPCYGDKDKRRHCFATWKNISGSIEIVK | 45 |
| | QGCWLDDINCYDRTDCVEKKDSPEVYFCCCEGNMCNEKFSYFPEMEVTQPTSNP | |
| | VTPKPP | |
| TIGIT | MMTGTIETTGNISAEKGGSIILQCHLSSTTAQVTQVNWEQQDQLLAICNADLGW | 46 |
| | HISPSFKDRVAPGPGLGLTLQSLTVNDTGEYFCIYHTYPDGTYTGRIFLEVLESSVAE | |
| | HGARFQIP | |
| FCGR2B | TPAAPPKAVLKLEPQWINVLQEDSVTLTCRGTHSPESDSIQWFHNGNLIPTHTQPS | 47 |
| | YRFKANNNDSGEYTCQTGQTSLSDPVHLTVLSEWLVLQTPHLEFQEGETIVLRCHS | |
| | WKDKPLVKVTFFQNGKSKKFSRSDPNFSIPQANHSHSGDYHCTGNIGYTLYSSKPV | |
| | TITVQAP | |
| FCGR1 | QVDTTKAVITLQPPWVSVFQEETVTLHCEVLHLPGSSSTQWFLNGTATQTSTPSYR | 48 |
| | ITSASVNDSGEYRCQRGLSGRSDPIQLEIHRGWLLLQVSSRVFTEGEPLALRCHAW | |
| | KDKLVYNVLYYRNGKAFKFFHWNSNLTILKTNISHNGTYHCSGMGKHRYTSAGISV | |
| | TVKELFPAPVLNASVTSPLLEGNLVTLSCETKLLLQRPGLQLYFSFYMGSKTLRGRNT | |
| | SSEYQILTARREDSGLYWCEAATEDGNVLKRSPELELQVLGLQLPTPVWFH | |
| 2B4 | CQGSADHVVSISGVPLQLQPNSIQTKVDSIAWKKLLPSQNGFHHILKWENGSLPSN | 49 |
| | TSNDRFSFIVKNLSLLIKAAQQQDSGLYCLEVTSISGKVQTATFQVFVFESLLPDKVE | |
| | KPRLQGQGKILDRGRCQVALSCLVSRDGNVSYAWYRGSKLIQTAGNLTYLDEEVDI | |
| | NGTHTYTCNVSNPVSWESHTLNLTQDCQNAHQEFRFWP | |
| LAIR1 | QEEDLPRPSISAEPGTVIPLGSHVTFVCRGPVGVQTFRLERESRSTYNDTEDVSQAS | 50 |
| | PSESEARFRIDSVSEGNAGPYRCIYYKPPKWSEQSDYLELLVKETSGGPDSPDTEPG | |
| | SSAGPTQRPSDNSHNEHAPASQGLKAEHLY | |
| CD5 | RLSWYDPDFQARLTRSNSKCQGQLEVYLKDGWHMVCSQSWGRSSKQWEDPSQ | 51 |
| | ASKVCQRLNCGVPLSLGPFLVTYTPQSSIICYGQLGSFSNCSHSRNDMCHSLGLTCL | |
| | EPQKTTPPTTRPPPTTTPEPTAPPRLQLVAQSGGQHCAGVVEFYSGSLGGTISYEA | |
| | QDKTQDLENFLCNNLQCGSFLKHLPETEAGRAQDPGEPREHQPLPIQWKIQNSSC | |
| | TSLEHCFRKIKPQKSGRVLALLCSGFQPKVQSRLVGGSSICEGTVEVRQGAQWAAL | |
| | CDSSSARSSLRWEEVCREQQCGSVNSYRVLDAGDPTSRGLFCPHQKLSQCHELWE | |
| | RNSYCKKVFVTCQDPNP | |
| TWEAKR | EQAPGTAPCSRGSSWSADLDKCMDCASCRARPHSDFCLGCAAAPPAPFRLLWP | 52 |

In some embodiments, the extracellular portions of the chimeric polypeptides disclosed herein (e.g., IL-9 switch receptors) having at least 80% sequence identity, such as, at least 80%, at least 85%, at least 90%, at least 95%, at least 96%, at least 97%, at least 98%, or 99% sequence identity to a sequence selected from the group consisting of SEQ ID NOS: 7-52 in the Sequence Listing. In some embodiments, the extracellular portion includes an amino acid sequence having at least 90% sequence identity to a sequence selected from the group consisting of SEQ ID NOS: 7-52. In some embodiments, the extracellular portion includes an amino acid sequence having at least 95% sequence identity to a sequence selected from the group consisting of SEQ ID NOS: 7-52. In some embodiments, the extracellular portion includes an amino acid sequence having about 100% sequence identity to a sequence selected from the group consisting of SEQ ID NOS: 7-52. In some embodiments, the extracellular portion includes an amino acid sequence having a sequence selected from the group consisting of SEQ ID NOS: 7-52, wherein one, two, three, four, or five of the amino acid residues in any one of the SEQ ID NOS: 7-52 is substituted by a different amino acid residue.

### Transmembrane Domain (TMD)

As outlined above, the chimeric switch receptors of the disclosure also include a transmembrane domain that joins the extracellular portion and the intracellular portion of the chimeric receptor.

The transmembrane domain is a region, which can be generally hydrophobic, and crosses the cell membrane. This domain can be positioned such that it directly or indirectly connects, or joins, the extracellular portion of the chimeric switch receptor to the intracellular portion of the chimeric switch receptor. This includes, but is not limited to recombinant fusions, covalent bonds, disulfide bonds, ionic bonds, hydrogen bonds, electrostatic bonds, and the like. Transmembrane domains may be a hydrophobic alpha helix that spans the cell membrane. The transmembrane domain associated with the endodomain is commonly used. However, in some embodiments, the transmembrane domain of TNFR1 is used in combination with extracellular portions from the TNF superfamily (e.g., DCR2, TNFRSF1, etc.) to stabilize the receptor structure.

The transmembrane domain can have any length. In some embodiments, the transmembrane domain includes 1 amino acid or 10 amino acids or 20 amino acids or 50 amino acids or 60 amino acids or 70 amino acids or 80 amino acids or 100 amino acids or 120 amino acids or 140 amino acids or 160 amino acids or 180 amino acids or 200 amino acids or 250 amino acids or 300 amino acids or any number therebetween.

In some embodiments, the transmembrane domain is selected from the transmembrane domain of IL-9, IL-7ra, IL-2rb, and TNFR1. Exemplary amino sequences of transmembrane domains for use herein are shown in Table 2.

TABLE 2
Transmembrane domain sequences

| Trans- | | SEQ |
|---|---|---|
| membrane | | ID |
| domain | Amino acid sequence | NO |
| IL-9 | LIPPWGWPGNTLVAVSIFLLLTGPTYLLFKLSPR | 53 |
| IL-7ra | PILLTISILSFFSVALLVILACVLW | 54 |
| IL-2rb | IPWLGHLLVGLSGAFGFIILVYLLI | 55 |
| TNFR1 | VLLPLVIFFGLCLLSLLFIGLMY | 56 |

In some embodiments, the transmembrane domain comprises the transmembrane domain of IL-9. In some embodiments, the transmembrane domain comprises the amino acid sequence of LIPPWGWPGNTLVAVSIFLLLTGPTYLLFKLSPR (SEQ ID NO: 53).

In some embodiments, the transmembrane domain comprises the transmembrane domain of TNFR1. In some embodiments, the transmembrane domain comprises the amino acid sequence of VLLPLVIFFGLCLLSLLFIGLMY (SEQ ID NO: 56).

In some embodiments, the transmembrane domain includes an amino acid sequence exhibiting at least 70%, at least 75%, at least 80%, at least 85%, at least 90%, at least 95%, at least 96%, at least 97%, at least 98%, at least 99% sequence identity to one or more of SEQ ID NOS: 53-56 in the Sequence Listing. In some embodiments, the transmembrane domain includes an amino acid sequence having at least 90% sequence identity to a sequence selected from the group consisting of SEQ ID NOS: 53-56. In some embodiments, the transmembrane domain includes an amino acid sequence having at least 95% sequence identity to a sequence selected from the group consisting of SEQ ID NOS: 53-56. In some embodiments, the transmembrane domain includes an amino acid sequence having about 100% sequence identity to one or more of SEQ ID NOS: 53-56. In some embodiments, the transmembrane domain includes an amino acid sequence having a sequence selected from the group consisting of SEQ ID NOS: 53-56, wherein one, two, three, four, or five of the amino acid residues in any one of the SEQ ID NOS: 53-56 is substituted by a different amino acid residue.

### Endodomain

In some embodiments, the endodomain is responsible for receptor clustering/dimerization after antigen binding and for initiation of signal transduction to the cell.

As outlined above, the chimeric receptors of the disclosure include an endodomain of an IL-9 receptor.

In some embodiments, the amino acid sequence for the IL-9 receptor endodomain is as follows in SEQ ID NO: 57 below:

VKRIFYQNVPSPAMFFQPLYSVHNGNFQTWMGAHGAGVLLSQDCAGTPQG
ALEPCVQEATALLTCGPARPWKSVALEEEQEGPGTRLPGNLSSEDVLPAG
CTEWRVQTLAYLPQEDWAPTSLTRPAPPDSEGSRSSSSSSSSNNNNYCAL
GCYGGWHLSALPGNTQSSGPIPALACGLSCDHQGLETQQGVAWVLAGHCQ
RPGLHEDLQGMLLPSVLSKARSWTF

In other embodiments, amino acid numbers 292 to 521 of IL-9Rα (NCBI REFSEQ: NP_002177.2) can be used.

Alternatively, a truncated fragment of said endodomain of the IL-9 receptor chain also may be used. For example, the truncated fragment comprises up to 250 amino acids, or is 50 to 200 amino acids or 80 to 150 amino acids of the ILR cytoplasmic domain.

As described supra, one aspect of the present disclosure relates to a recombinant nucleic acid encoding a chimeric receptor including: (a) an extracellular portion comprising a binding domain of an endogenous inhibitory receptor; (b) an intracellular portion comprising an endodomain of an IL-9 receptor linked to a BOX½ common gamma chain domain; (c) a transmembrane domain that joins the extracellular portion and the intracellular portion. Extracellular portions comprising binding domains of inhibitory receptors are described above and are useful in the chimeric receptors described infra as well. In this category of chimeric receptors, signaling through the IL-9 endodomain requires recruitment of the common gamma chain. In some embodiments, the binding domains of the extracellular portion of the chimeric receptors described herein are not able to naturally recruit the common gamma chain in order to elicit signaling through the IL-9 endodomain of the chimeric receptor. Accordingly, in these embodiments, the endodomain of the IL-9 receptor can be linked to BOX½ domain. Receptors of the common gamma chain family contain two regions at the cytoplasmic tail, termed Box 1 and Box 2. These domains are critical for the association of JAKs with the receptor (see for example, Murakami M, Narazaki M, Hibi M, Yawata H, Yasukawa K, Hamaguchi M, Taga T, Kishimoto T (1991) Critical cytoplasmic region of the interleukin 6 signal transducer gp130 is conserved in the cytokine receptor family. Proc Natl Acad Sci USA 88: 11349-11353). Generally, the Box 1 domain contains a proline-rich segment of amino acid residues, and the Box2 domain contains a hydrophobic segment of amino acid residues.

In some embodiments, the chimeric receptor comprises a BOX½ domain of the following amino acid sequence of SEQ ID NO: 58

ERTMPRIPTLKNLEDLVTEYHGNFSAWSGVSKGLAESLQPDYSERLCLVS
EIPPKGGALGEGPGASPCNQHSPYWAPPCYTLKPET

### Linkers

The nucleic acid encoding the chimeric receptor can further comprise a linker between any of the portions or domains described above. As used herein, the term “linker” generally refers to an oligopeptide or polypeptide that functions to link a one region of a nucleic acid to another region of a nucleic acid. The spacer or linker may comprise up to 300 amino acids, 0-100 amino acids, 25-50 amino acids, 10-15 amino acids, for example.

Linkers useful in the chimeric receptors described herein include those in Table 3 below.

| Linker | Amino acid sequence | SEQ ID NO |
|---|---|---|
| Gly Ser Linker | GGGGSGGGGSGGGGS | 59 |
| Q-Pro Linker | QPQPQPQPQPQP | 60 |
| K-Pro Linker | KPKPKPKPKPKP | 61 |

In some embodiments, the linker is a Gly Ser Linker. In some embodiments, the linker comprising the amino acid sequence GGGGSGGGGSGGGGS (SEQ ID NO:59).

In some embodiments, the linker is a Q-Pro Linker. In some embodiments, the linker comprising the amino acid sequence QPQPQPQPQPQP (SEQ ID NO:60).

In some embodiments, no linker is present.

In some embodiments, the linker can be a 2A self-cleaving peptide. 2A peptide are a class of 18-22 amino acid peptides, which can result in ribosomal skipping during translation of a protein in a cell. In some embodiments, a linker, such as a 2A peptide, can be included to link the nucleic acid region encoding the chimeric receptor of the disclosure to another nucleic acid region encoding a chimeric antigen receptor (CAR). In some embodiments, the CAR is anti-HER2 CAR (4D5).

### Signal Sequence

A “signal sequence” can also included at the beginning of the coding sequence of the chimeric receptors disclosed herein. This sequence encodes a signal peptide, N-terminal to the mature polypeptide, that directs the host cell to translocate the polypeptide. Translocation signal sequences can be found associated with a variety of proteins native to eukaryotes and prokaryotes, and are often functional in both types of organisms.

An exemplary signal sequence that can be used in the chimeric receptors herein includes the amino acid sequence of: MAAPALSWRLPLLILLLPLATSWASA (SEQ ID NO: 62)

### Nucleic Acid Molecules

Provided herein are various nucleic acid molecules including nucleotide sequences encoding the chimeric receptors of the disclosure. In some embodiments, expression cassettes and expression vectors contain these nucleic acid molecules operably linked to heterologous nucleic acid sequences such as, for example, regulatory sequences which allow in vivo expression of the receptor in a host cell.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence encoding a chimeric polypeptide including: (a) an extracellular portion comprising a binding domain of an endogenous cytokine receptor; (b) a transmembrane domain; and (c) an intracellular portion comprising an endodomain of an IL-9 receptor.

In one embodiment, the nucleic acid molecule includes a sequence encoding a chimeric polypeptide including: (a) a binding domain of IL-21; (b) a transmembrane domain of IL-9; and (c) an endodomain of an IL-9 receptor.

In one embodiment, the nucleic acid molecule includes a sequence encoding a chimeric polypeptide including: (a) a binding domain of IL-4; (b) a transmembrane domain of IL-9; and (c) an endodomain of an IL-9 receptor.

In one embodiment, the nucleic acid molecule includes a sequence encoding a chimeric polypeptide including: (a) a binding domain of IL-15Ra; (b) a transmembrane domain of IL-9; and (c) an endodomain of an IL-9 receptor.

In one embodiment, the nucleic acid molecule includes a sequence encoding a chimeric polypeptide including: (a) a binding domain of IL-10Ra; (b) a transmembrane domain of IL-9; and (c) an endodomain of an IL-9 receptor.

In some embodiments, provided herein is a nucleic acid molecule including a nucleotide sequence encoding a chimeric polypeptide including: (a) an extracellular portion comprising a binding domain of an endogenous inhibitory receptor; (b) a transmembrane domain; and (c) an intracellular portion comprising an endodomain of an IL-9 receptor linked to a BOX½ common gamma chain domain.

The orientation of the endodomain of the IL-9 receptor linked to a BOX ½ common gamma chain domain may be altered depending upon desired structure and function. For example, the intracellular portion can comprise, in an N-to C-terminal direction, an endomain of an IL-9 receptor, a linker, and a BOX ½ common gamma chain domain. Alternatively, the intracellular portion can comprise, in an N-to C-terminal direction, a BOX ½ common gamma chain domain, a linker, and an endomain of an IL-9 receptor.

In one embodiment, the nucleic acid molecule includes a sequence encoding a chimeric polypeptide including: (a) a binding domain of Fas; (b) a transmembrane domain of IL-9; and (c) an endodomain of an IL-9 receptor linked to a BOX½ common gamma chain domain.

In some embodiments, provided herein is a nucleic acid molecule including a nucleotide sequence encoding a chimeric polypeptide including: (a) an extracellular portion comprising a binding domain of an endogenous inhibitory receptor linked to an agent specific for the common gamma chain; (b) a transmembrane domain; and (c) an intracellular portion comprising an endodomain of an IL-9 receptor.

The orientation of the extracellular portion comprising a binding domain of an endogenous inhibitory receptor linked to an agent specific for the common gamma chain may be altered depending upon desired structure and function. For example, the extracellular portion can comprise, in an N-to C-terminal direction, a binding domain of an endogenous inhibitory receptor, a linker, and an agent specific for the common gamma chain. Alternatively, the extracellular portion can comprise, in an N-to C-terminal direction, an agent specific for the common gamma chain, a linker, and a binding domain of an endogenous inhibitory receptor.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into a subject. As such, in some embodiments, an expression cassette of the disclosure include a coding sequence for the chimeric polypeptide as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into a cloning vector or an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector. In some embodiments, the nucleotide sequence is incorporated into a cloning vector.

As described above in realtion to linkers, the nucleic acid sequence encoding a chimeric receptor as described herein can also encode a CAR. Each may each be provided on separate expression vectors, each nucleic acid sequence being operably linked to one or more expression control elements to achieve expression of the CAR and chimeric receptor in the target cell, the vectors being co-transfected into the target cell. Alternatively, the nucleic acid sequences encoding the CAR and the chimeric receptor may each be provided on a single vector each nucleic acid sequence under the control of one or more expression control elements to achieve expression of the associated nucleic acid sequence. Alternatively, both nucleic acid sequences may be under the control of a single promoter with intervening or downstream control elements that facilitate co-expression of the two sequences from the vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 80%, 90%, 95%, 96%, 97%, 98%, 99%, or 100% sequence identity to a chimeric receptor disclosed herein. In some embodiments, provided herein are nucleic acid molecules encoding a polypeptide with an amino acid sequence having at least about 80%, 90%, 95%, 96%, 97%, 98%, 99%, or 100% sequence identity to any one of SEQ ID NOS: 63-203 as identified in the Sequence Listing.

The nucleic acid sequences encoding the chimeric receptors can be optimized for expression in the host cell of interest. For example, the G-C content of the sequence can be adjusted to average levels for a given cell, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into a subject. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding a chimeric receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to hercin as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans of ordinary skill are able to select and design expression systems suitable and functional in a selected engineered cell. If further guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

### Recombinant cells and Cell Cultures

The nucleic acid of the present disclosure can be introduced into a host cell, such as, for example, a human T lymphocyte, to produce a host cell containing the recombinant nucleic acid molecule. Accordingly, some embodiments of the disclosure relate to methods for making a host cell, including (a) providing a cell capable of protein expression and (b) contacting the provided cell with a recombinant nucleic acid of the disclosure.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant cell as a mini-circle expression vector for transient expression. Accordingly, in some embodiments, the nucleic acid molecule is maintained and replicated in the recombinant cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene-and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively casy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. These cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a prokaryotic cell including a recombinant nucleic acid as disclosed herein. In some embodiments, the recombinant prokaryotic cell includes a recombinant nucleic acid which is a cloning vector. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an cosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a subject. In some embodiments, the subject is a human patient.

In one embodiment, the cell expressing the recombinant nucleic acid molecule described herein is a T-cell which has been modified to surface express a chimeric antigen receptor (a‘CAR-T’ cell). As used herein, a CAR-T cell may be engineered to express a chimeric receptor of the present disclosure. CARs useful in the practice of the present disclosure can be prepared in accordance with principles well known in the art. See e.g., Eshhaar et a/. U.S. Pat. No. 7,741,465 B1 issued Jun. 22, 2010; Sadelain, et al (2013) Cancer Discovery 3 (4): 388-398; Jensen and Riddell (2015) Current Opinions in Immunology 33:9-15; Gross, et al. (1989) PNAS (USA) 86 (24): 10024-10028; Curran, et al. (2012) J Gene Med 14 (6): 405-15. Examples of commercially available CAR-T cell products that may be modified to incorporate a chimeric receptor of the present disclosure include axicabtagene ciloleucel (marketed as Yescarta® commercially available from Gilead Pharmaceuticals) and tisagenlecleucel (marketed as Kymriah® commercially available from Novartis).

In some embodiments, the recombinant cell further includes a first and a second nucleic acid molecule as disclosed herein, wherein the first nucleic acid molecule and the second nucleic acid molecule do not have the same sequence. In some embodiments, the recombinant cell further includes a first and a second chimeric polypeptide as disclosed herein, wherein the first chimeric polypeptide and the second chimeric polypeptide do not have the same sequence. In some embodiments, the first second chimeric polypeptide is a CAR. In some embodiments, the first chimeric polypeptide modulates the expression and/or activity of the second chimeric polypeptide.

In some embodiments, the recombinant cell further includes an expression cassette or vector encoding a protein of interest operably linked to a promoter, wherein expression of the protein is modulated by the chimeric receptor's transcriptional effector. In some embodiments, the protein of interest is heterologous to the recombinant cell. In some embodiments, the heterologous protein is one that is not normally found in the cell, e.g., not normally produced by the cell. In some embodiments, the expression vector encodes a copy of a protein that is already present in the cell. Exemplary types of proteins suitable for use with the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporters.

In another aspect, provided herein are compositions of cells comprising an expression vector described herein. Cell cultures including at least one host cell as disclosed herein, and a culture medium are also contemplated. Generally, the culture medium can be any suitable culture medium for culturing the cells described herein. Techniques for transforming a wide variety of the above-mentioned cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, host cells, and/or polypeptides (i.e., chimeric receptors) of the disclosure can be incorporated into compositions, including pharmaceutical compositions. Such compositions include one or more of the recombinant nucleic acids, host cells, and/or polypeptides (i.e., chimeric receptors) as disclosed herein. The composition can also contain a pharmaceutically acceptable excipient, e.g., a carrier.

Pharmaceutical compositions suitable for injectable use include sterile aqueous solutions (where water soluble) or dispersions and sterile powders for the extemporancous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the chimeric receptors of the disclosure can also be administered by transfection or infection using methods known in the art, including but not limited to the methods described in McCaffrey et al. (Nature 418:6893, 2002), Xia et al. (Nature Biotechnol. 20:1006-10, 2002), or Putnam (Am. J. Health Syst. Pharm. 53:151-60, 1996, erratum at Am. J. Health Syst. Pharm. 53:325, 1996).

As described in greater detail below, in some embodiments, the host cells of the disclosure can be formulated for administration to a subject using techniques known to the skilled artisan. For example, formulations comprising populations of recombinant cells can include pharmaceutically acceptable excipient(s). Excipients included in the formulations will have different purposes depending, for example, on the recombinant cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising recombinant cells can have been prepared and cultured in the absence of non-human components, e.g., in the absence of animal serum. A formulation can include one population of recombinant cells, or more than one, such as two, three, four, five, six or more populations of recombinant cells.

Formulations comprising population(s) of recombinant cells can be administered to a subject using modes and techniques known to the skilled artisan. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Devices useful for parenteral injection of infusion of the formulations can be used to effect such administration.

### METHODS OF THE DISCLOSURE

### Methods for Modulating an Activity of a Cell

In one aspect, provided herein are methods for modulating an activity of an immune cell. The methods involve administering, to an immune cell, the recombinant nucleic acid as described herein. One skilled in the art upon reading the present disclosure will appreciate that the disclosed methods can be carried out in vivo, ex vivo, or in vitro.

Non-limiting exemplary cellular activities that can be modulated using the methods provide herein include, but are not limited to, gene expression, proliferation, apoptosis, non-apoptotic death, differentiation, dedifferentiation, migration, secretion of a gene product, cellular adhesion, and cytolytic activity.

In some embodiments, the expression of a gene product of the cell is modulated.

In some embodiments, the gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T cell receptor (TCR) or a component thereof, a chimeric antigen receptor (CAR), a toxin, a toxin-derived protein, a transcriptional effector, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In cases where expression of a chemokine or a cytokine is modulated, in some embodiments, the expression of cytokines and chemokines is modulated such that there is an increase in type-1 polarization following ligand stimulation relative to controls. Exemplary cytokine and chemokines that can be modulated include, without limitation, FGF2, GMCSF, IFNa, IFNg, IL-10, IL-17, IL-12, IL-2, IL-3, IL-6, IP-10, MIP1a, and RANTES.

Methods of measuring levels of cytokines and chemokines are known in the art. Levels of cytokines and chemokines can be measured, for example, by enzyme-linked immunosorbent assay (ELISA), bead based systems (e.g. Luminex), the Cytokine Bead Array (Pharmingen) and array-based systems (e.g., EMD Biosciences' ProteoPlex).

In some embodiments, the modulation of an activity of an immune cell can involve alteration of cell signaling events within the cell. As described herein, the IL-9 receptor endodomain signals through JAK1 and JAK3 to activate various developmental pathways including STAT1, STAT3, STAT5, MAPK, and PI3K/AKT pathways. Activation of STAT family members through ligand mediated phosphorylation is believed to confer advantages in effector function, polarization and proliferation to T cells. This, in some embodiments, administration of the recombinant nucleic acid encoding the chimeric switch receptor described herein induces phosphorylation of STAT1, STAT3, and/or STAT5 when stimulated with ligand. Methods for measuring the phosphorylation status of one or more proteins is known in the art and includes, for example, western blot and phospho flow cytometry as described in the Examples herein.

Cell death (e.g., apoptosis and non-apoptotic cell death) can also be modulated by the methods described herein. In some embodiments, administration of the recombinant nucleic acid encoding the chimeric switch receptor described herein can result in increased cell death of a target cell. By way of example, real-time cytotoxicity assays can be used to analyze whether or not cells expressing a chimeric switch receptor of the present disclosure show increased cytotoxicity when exposed to ligand. In some embodiments, cells expressing a chimeric switch receptor of the present disclosure demonstrate improved killing of target cells when stimulated with ligand over unstimulated controls.

### Methods of Treatment

Administration of any one of the therapeutic compositions described herein, e.g., chimeric receptors, nucleic acids, host cells, and pharmaceutical compositions, can be used to treat patients for relevant diseases, such as cancers, autoimmune diseases, and infections. In some embodiments, the recombinant nucleic acids, host cells, and pharmaceutical compositions described herein can be incorporated into therapeutic agents for use in methods of treating or aiding in the treatment of a subject who has, who is suspected of having, or who may be at high risk for developing one or more diseases.

One aspect of the present disclosure is directed to a method of treating a subject that involves administering, to the subject, a chimeric switch receptor of the present disclosure or a cell expressing the recombinant nucleic acid of the present disclosure.

In one embodiment, the subject is treated for cancer. A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma. In particular, treatment of melanoma, kidney cancer (e.g. renal carcinoma) or bladder cancer is contemplated.

In some embodiments the cancer is an EBV or HPV positive cancer.

In one embodiment, the subject is treated for autoimmune disease. Exemplary autoimmune diseases include Crohn's disease and Multiple Sclerosis.

In one embodiment, the subject is treated for infection. An infection may be any infection or infectious disease, e.g. bacterial, viral, fungal, or parasitic infection. In some embodiments it may be particularly desirable to treat chronic/persistent infections, e.g. where such infections are associated with T cell dysfunction or T cell exhaustion. It is well established that T cell exhaustion is a state of T cell dysfunction that arises during many chronic infections (including viral, bacterial and parasitic), as well as in cancer (Wherry Nature Immunology Vol.12, No.6, p492-499, June 2011).

Examples of bacterial infections that may be treated include infection by Bacillus spp., Bordetella pertussis, Clostridium spp., Corynebacterium spp., Vibrio chloerac, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa. For example, the bacterial infection may be sepsis or tuberculosis. Examples of viral infections that may be treated include infection by Epstein-Barr virus, influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus.

Examples of fungal infections that may be treated include infection by Alternaria sp, Aspergillus sp, Candida sp and Histoplasma sp. The fungal infection may be fungal sepsis or histoplasmosis. Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis

### Administration of Recombinant Cells to a Subject

In some embodiments, the methods of the disclosure involve administering an effective amount or number of the recombinants cells to a subject in need of such treatment. This administering step can be accomplished using any method of implantation delivery in the art. For example, the recombinant cells can be infused directly in the subject's bloodstream or otherwise administered to the subject.

In some embodiments, the methods disclosed herein include administering, which term is used interchangeably with the terms “introducing”, implanting “, and “transplanting”, recombinant cells into a subject, by a method or route that results in at least partial localization of the introduced cells at a desired site such that a desired effect(s) is/are produced. The recombinant cells or their differentiated progeny can be administered by any appropriate route that results in delivery to a desired location in the subject where at least a portion of the administered cells or components of the cells remain viable. The period of viability of the cells after administration to a subject can be as short as a few hours, e.g., twenty-four hours, to a few days, to as long as several years, or even the lifetime of the subject, i.e., long-term engraftment.

When provided prophylactically, the recombinant cells described herein can be administered to a subject in advance of a symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10₂ cells, at least 10³ cells, at least 5×10₃ cells, at least 10⁴ cells, at least 5×10₄ cells, at least 10⁵ cells, at least 2×10₅ cells, at least 53×10cells, at least 4×10₅ cells, at least 5×10₅ cells, at least 6×10₅ cells, at least 7×10₅ cells, at least 8×10₅ cells, at least 9×10₅ cells, at least 1×10₆ cells, at least 2×10₆ cells, at least 3×10₆ cells, at least 4×10₆ cells, at least 5×10₆ cells, at least 6×10₆ cells, at least 7×10₆ cells, at least 8×10₆ cells, at least 9×10₆ cells, or multiples thereof. The recombinant cells can be derived from one or more donors or can be obtained from an autologous source. In some embodiments, the recombinant cells are expanded in culture prior to administration to a subject in need thereof.

In some embodiments, the delivery of a recombinant cell composition (e.g., a composition including a plurality of recombinant cells according to any of the cells described herein) into a subject by a method or route results in at least partial localization of the cell composition at a desired site. A composition including recombinant cells can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection. For example, a population of recombinant cells are administered other than directly into a target site, tissue, or organ, such that it enters, the subject's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments of the disclosed methods, the subject is a mammal. In some embodiments, the mammal is a human.

### Additional Therapies

As discussed above, the recombinant cells, and pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antincoplastics.

Accordingly, in some embodiments, the disclosed treatment methods further include administering to the subject a second therapy. Generally, the second therapy can be any therapy known in the art. Non-limiting examples of therapies suitable for use in combination with the therapeutic compositions disclosed herein include chemotherapy, radiotherapy, immunotherapy, hormonal therapy, toxin therapy, and surgery. In some embodiments, the second therapy includes one or more additional therapeutic agents such as, for example, chemotherapeutics or anti-cancer agents or anti-cancer therapies. In some embodiments, the first therapy and the second therapy are administered together in the same composition. In some embodiments, the first therapy and the second therapy are administered in separate compositions. In some embodiments, the first therapy and the second therapy are administered at the same time. In some embodiments, the first therapy and the second therapy are administered sequentially. In some embodiments, the first therapy is administered before the second therapy. In some embodiments, the first therapy is administered after the second therapy. In some embodiments, the first therapy and the second therapy are administered in rotation.

### Systems and Kits

Also provided herein are kits including the recombinant nucleic acids, recombinant cells, or pharmaceutical compositions provided and described herein as well as written instructions for making and using the same. For example, provided herein, in some embodiments, are kits that include one or more of the following: (i) a recombinant nucleic acids as described herein, (ii) a recombinant cell as described herein, and (iii) a pharmaceutical composition as described herein. In some embodiments, the systems and/or kits of the disclosure further include one or more syringes (including pre-filled syringes) and/or catheters (including pre-filled syringes) used to administer one any of the provided recombinant nucleic acids, recombinant cells, or pharmaceutical compositions to a subject. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in a subject in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), are provided. An example of this embodiment is a kit that includes a web address where the instructions can be viewed and/or from which the instructions can be downloaded. As with the instructions, this means for obtaining the instructions can be recorded on a suitable substrate.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety to the same extent as if each individual publication or patent application was specifically and individually indicated to be incorporated by reference.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

Cloning of chimeric receptors. DNA encoding the binding domains of IL-2rb, IL-2ra, IL-4r, IL-7ra, IL-15ra, IL-21ra, TGF-beta R1, TGF-beta R2, IL-10ra, FAS, CTLA4, LAG3, TIM3, PD1, ILT2, ILT3, ILT4, ILT5, VEGF; the transmembrane domains of IL-9, IL-7ra and IL-2rb; the endodomain of the IL-9 receptor; the BOX ½ common gamma chain domain; and an agent specific for the common gamma chain are cloned, in various configurations, into mammalian expression vectors driven by a CMV or elongation factor (EF)-1 promoter. The vector contains a mammalian selection cassette.

Similarly, DNA encoding the binding domains of IL-2rb, IL-2ra, IL-4r, IL-7ra, IL-15ra, IL-21ra, TGF-beta R1, TGF-beta R2, IL-10ra, FAS, CTLA4, LAG3, TIM3, PD1, ILT2, ILT3, ILT4, ILT5, VEGF; the transmembrane domains of IL-9, IL-7ra and IL-2rb; the endodomain of the IL-9 receptor; the BOX ½ common gamma chain domain; and an agent specific for the common gamma chain are cloned, in various configurations, into lentiviral vectors.

### Example 2

Lentivirus expression. Lentivirus is produced as previously described in [Tiscornia G. et al. Nature Protocols 27 Jun. 2006; doi: 10.1038/nprot.2006.37].

### Example 3

Assessment of surface expression of chimeric receptors. Mammalian expression constructs encoding for chimeric receptors are transfected into 293 cells using methods well known in the art such as lipofection 2000 (Invitrogen) or electroporation. 24 to 48 hours later, cell surface expression is assessed by flow cytometry using fluorescently labeled antibodies specific for the ectodomains chimeric receptors. An example would be FITC labeled anti-ILAR antibody for 293 cells transduced with construct 73.

Alternatively, Jurkat T cell are transduced with purified lentivirus expressing the chimeric receptors of interest at an MOI of 20. 24 to 48 hours later, cell surface expression is assessed by flow cytometry using fluorescently labeled antibodies specific for the ectodomains chimeric receptors.

### Example 4

Ligand stimulation and STAT5 activation. Transfected 293 cells and virally transduced Jurkat T cells are stimulated with individual ligands to analyze the ability of the ligands to bind the chimeric receptors and induce STAT5 activation. Briefly, after stimulation with ligand for various time points, cells are lysed and protein is collected. The phosphorylation status of STAT5 is analyzed by Western Blot using phosphor STAT5 specific antibodies and assessed relative to a negative control comprising an empty vector. STAT5 phosphorylation status of ligand activated cells relative to empty vector controls is also assessed by flow cytometry as previously described. (http://rhlccflow.facilities.northwestern.edu/files/2011/09/intracellular-phospho-protein-staining.pdf)

### Example 5

STAT5 reporter assay. STAT5 reporter assays are done in 293 cells for activated JAK1-3 induced activity. Cells are seeded in a 48 well plate. The next day, cells are transfected by Lipofectamine 2000 (Invitrogen) with the STAT5-Luciferase vector and an internal control plasmid together with other plasmids expressing genes of interest. 24 hours later, the cells are stimulated with appropriate ligand for various time points, then lysed and subjected to internal control fluorescence and luciferase luminescence measurement using a plate reader. The reporter gene activity is shown after being normalized against internal control readings.

### Example 6

Ligand stimulation and cytokine expression. Jurkat T cell are transduced with purified lentivirus expressing the chimeric receptors of interest. 24 to 48 hours later, cells are stimulated with appropriate ligands for various time points from 0, 15, 30 45 and 60 minutes. Cells are then collected for analysis of expression of IFNγ, IL-4, IL-5, IP-10, IL-2, MIP1α, MIP1β, and TNFα using intracellular flow cytometry. Supernatants from the culture are collected analyzed for the expression of 30 cytokines and chemokines (Thermo Fisher) (https://www.thermofisher.com/order/catalog/product/LHC6003M #/LHC6003M) by Luminex analysis.

### Example 7

Cytotoxicity Assays. The ability of the recombinant nucleic acids described herein to stimulate CTL cytotoxicity can be measured by methods known to the skilled person. Cytotoxicity of a T cell to a given target cell can be investigated, for example, using any of the methods reviewed in Zaritskaya et al. Expert Rev Vaccines (2011), 9(6): 601-616, hereby incorporated by reference in its entirety. Additionally, the ability of the chimeric cytokine receptor to enhanced cell killing of a GPC3 CAR is assessed by measuring cellular killing of cell line targets in real time by electrical impedance as described in (https://www.ncbi.nlm.nih.gov/pmc/articles/PMC5834184/). GPC3 CAR (scFv-41BB-CD3ζ) expressing Lentivirus will be purchased from Creative Biolabs (CAR-M0158-YC) and used to transduce T cells. Briefly, T cells will be isolated from the PBMC fraction of peripheral blood by negative selection. Cells will be stimulated with TransAct (Miltenyi) for 72 hours in the presence of IL-2 prior to Lentiviral transduction. Cells will be incubated for 48 hours and CAR expressing cells sorted by FACS. The CAR positive fraction will then be transduced with the chimeric cytokine receptor constructs. Cell surface expression of the chimeric cytokine receptor will be assessed 48 hours post transduction, by flow cytometry. Cells will be stimulated with the appropriate ligand for an additional 48 hours, washed and cultured with HEPG2 target cells on XCellegence plates (https://www.agilent.com/en/product/cell-analysis/real-time-cell-analysis/ rtca-analyzers/xcelligence-rtca-mp-multiple-plates-741230). Transduced T cells and target cells will be cocultured for 96 hours. Target killing will be measured in real time according to the manufacturer's protocol.

### Example 8

### Materials and Methods for Examples 9-12

Cloning of chimeric receptors. DNA encoding the binding domains of various endogenous cytokine and inhibitory receptors; the transmembrane domains of IL-9 or TNFR1; the endodomain of the IL-9 receptor; and/or the BOX ½ common gamma chain domain; were cloned, in various configurations, into pTRPE backbone lentiviral transfer plasmid. Some receptors were co-expressed with anti-HER2 CAR (4D5) via a 2A linker sequence. The 4D5 amino acid sequence is as follows:

(SEQ ID NO: 204)
MDFQVQIFSFLLISASVIMSRGDIQMTQSPSSLSASVGDRVTITCRASQD
VNTAVAWYQQKPGKAPKLLIYSASFLYSGVPSRFSGSRSGTDFTLTISSL
QPEDFATYYCQQHYTTPPTFGQGTKVEIKRTGSTSGSGKPGSGEGSEVQL
VESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGF
GGAVHTRGLDFACDIYIWAPLAGTCGVLLLSLVITLYCKRGRKKLLYIFK
QPFMRPVQTYAMDVWGQGTLVTVSSTTTPAPRPPTPAPTIASQPLSLRPE
ACRPAATQEEDGCSCRFPEEEEGGCELRVKFSRSADAPAYKQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR*

Similarly, DNA encoding the binding domains of IL-21R, IL-15ra, IL-4ra, IL-10ra, IL-7ra, TWEAKR, CTLA4, TIM3, LAG3, PD1, DCR1, CD40, Fas, Dr4, TNFRSF1B, TGFBR2, TIGIT, 2B4; the transmembrane domains of IL-9 and TNER1; the endodomain of the IL-9 receptor; the BOX ½ common gamma chain domain; were cloned, in various configurations, into lentiviral vectors.

Assessment of surface expression of chimeric receptors. Lentiviral transfer plasmids encoding for chimeric receptors were transfected into 293 cells using Lipofectamine 3000 and 500 ng of lentiviral expression plasmids. 24 hours later, cell surface expression was assessed by flow cytometry using fluorescently labeled antibodies specific for the ectodomains of the chimeric receptors. Expression of chimeric receptors on primary human T cells was measured by transducing primary human T cells with a dilution series of lentiviral supernatants and analyzed by flow cytometry 72 hours later.

Lentivirus. Lentiviruses for transduction of primary human CD3+ T cells were produced in 293T cells by lipofection (Lipofectamine 3000, Thermo Fisher Scientific) of transfer and packaging plasmids, and purified by ultracentrifugation. T cells activated with human CD3/CD28 Dynabeads (3:1 beads to cell ratio) in the presence of recombinant IL-7 and IL-15 were infected with lentiviruses one day after activation, de-beaded on Day 3, and expanded until Day 5.

Flow Cytometry. Flow cytometric detection of chimeric cytokine receptors and chimeric antigen receptors was performed by incubating lentivirally transduced primary human T cells with receptor-specific antibodies for 20 minutes in room temperature in the dark followed by acquisition of at least 10,000 events on FACSymphony A3 flow cytometer (BD Biosciences). Data was analyzed with FlowJo software (BD Biosciences). Transiently transfected 293T cells were treated similarly to detect chimeric cytokine receptors and chimeric antigen receptors 24 hours after lipofection (Lipofectamine 3000, Thermo Fisher Scientific) of 500 ng of lentiviral expression plasmids.

pSTAT Detection. In phosphoflow experiments, transduced human T cells were stimulated by addition of ligands for 30 minutes at 37° C., and the reaction was terminated by fixation with 1.5% paraformaldehyde (PFA) for 15 min at room temperature with agitation. Cells were washed and permeabilized with ice-cold 100% methanol for 60 minutes on ice or stored at −80° C. overnight. Cells were washed with FACS buffer before staining with pSTAT antibodies (Thermo Fisher Scientific) for 1 h at 4° C. in the dark. Cells were washed and analyzed on FACSymphony A3 flow cytometer. Data represent the mean fluorescence intensity (MFI).

xCELLigence Real-Time Cell Analysis. Tumor cell killing was assessed using the xCELLigence Real-Time Cell Analysis (RTCA) Analyzer (Agilent). SKOV-3 human ovarian adenocarcinoma tumor cells were seeded on a 96-well xCELLigence E-Plate at 10,000 cells per well. Twenty-four hours later, transduced T cells with or without 48 hours of pre-incubation with ligands for switch receptors were added in triplicate at various effector-to-target ratios with or without continued ligand stimulation. At the end of the assay, supernatant from each experimental well of the E-Plate was harvested, centrifuged to remove debris, then immediately frozen at −80C.

Cytokine Multiplex Analysis. Samples were analyzed for cytokines and chemokines using the Milliplex cytokine assay kit (Millipore) as per manufacturer's protocol. Briefly, samples were diluted 1:2.5 assay diluent buffer and loaded onto a Millipore Multiscreen BV 96-well filter plate. Serial dilutions of cytokine standards were prepared in parallel and added to the plate. Milliplex 42-Plex Cytokine beads were vortexed for 30 sec. and 25 ul was added to each well with culture supernatants. Samples were then incubated on a plate shaker at 600 rpm in the dark at room temperature for 2 hours. The plate was applied to a Millipore Multiscreen Vacuum Manifold, washed twice with 50 μl of assay buffer (PBS, pH7.4, 1% BSA, 0.05% Tween20, 0.05% sodium azide), and each well resuspended with 75 μl assay buffer. 25 μl of biotinylated Anti-Human Multi-Cytokine Reporter was added to each well. The plate was incubated on a plate shaker at 600 rpm in the dark at room temperature for 1.5 hours. Streptavidin-Phycoerythrin was diluted 1:12.5 in assay buffer, and then 25 μl was added directly to each well. The plate was incubated on a plate shaker at 600 rpm in the dark at room temperature for 30 minutes. 25 μl of stop solution (0.2% (v/v) formaldehyde in PBS, pH 7.4) was added to each well and incubated at room temperature for 5 minutes. The plate was then applied to the vacuum manifold and each well resuspended in 125 μl assay buffer and shaken for 1 minute. Assay plate was the transferred to the Bio-Plex Luminex 200 XYP instrument for analysis. Cytokine concentrations were calculated using Bio-Plex Manager 6.2 software with a 5 parameter curve fitting algorithm applied for standard curve calculations.

### Example 9

### Chimeric Switch Receptors Are Expressed on the Surface of Cells

In order to analyze whether the chimeric switch receptors were able to be correctly folded and expressed on the surface of cells, 293T cells were transiently transfected with lentiviral transfer plasmids encoding for the chimeric switch receptors. 24 hours later, cell surface expression was assessed by flow cytometry. The expression levels of various constructs are shown in Table 4 below.

TABLE 4

| | Ligand- | Trans- | Intra- | | % expression | % expression | % | % double- |
|---|---|---|---|---|---|---|---|---|
| Sequence | binding | membrane | cellular | | transduced | control | positive | positive |
| ID | domain | domain | domain | Linker | sample | sample | cells | cells |
| SEQ63 + | IL21R | IL9R | IL9R | N/A | 45.4 | 0.031 | | 45.369 |
| CAR+ | | | | | | | | |
| SEQ72 + | IL4Ra | IL9R | IL9R | N/A | 16.7 | 0.5 | | 16.2 |
| CAR+ | | | | | | | | |
| SEQ89 + | CTLA4 | IL9R | IL9R | G₄S | 28.8 | 0.72 | | 28.08 |
| CAR+ | | | | | | | | |
| SEQ90 + | CTLA4 | IL9R | IL9R | QP | 26.6 | 0.22 | | 26.38 |
| CAR+ | | | | | | | | |
| SEQ91 | TIM3 | IL9R | IL9R | G₄S | 21.5 | 3.42 | 18.08 | |
| SEQ93 | LAG3 | IL9R | IL9R | QP | 1.33 | 0.13 | 1.2 | |
| SEQ94 | LAG3 | IL9R | IL9R | G₄S | 3.35 | 2.25 | 1.1 | |
| SEQ95 + | PD1 | IL9R | IL9R | G₄S | 18.3 | 0.49 | | 17.81 |
| CAR+ | | | | | | | | |
| SEQ96 + | PD1 | IL9R | IL9R | QP | 12.2 | 0.21 | | 11.99 |
| CAR+ | | | | | | | | |
| SEQ109 | DCR1 | IL9R | IL9R | G₄S | 26 | 11.2 | 14.8 | |
| SEQ110 | DCR1 | IL9R | IL9R | QP | 17.6 | 7.66 | 9.94 | |
| SEQ111 | CD40 | IL9R | IL9R | G₄S | 24 | 1.42 | 22.58 | |
| SEQ112 | CD40 | IL9R | IL9R | QP | 19.2 | 0.31 | 18.89 | |
| SEQ113 + | Fas | IL9R | IL9R | G₄S | 22.9 | 1.1 | | 21.8 |
| CAR+ | | | | | | | | |
| SEQ114 + | Fas | IL9R | IL9R | QP | 24.9 | 1.03 | | 23.87 |
| CAR+ | | | | | | | | |
| SEQ115 | DR4 | IL9R | IL9R | G₄S | 54.5 | 34.7 | 19.8 | |
| SEQ116 | DR4 | IL9R | IL9R | QP | 38.3 | 21.4 | 16.9 | |
| SEQ123 | TNFRSF1B | IL9R | IL9R | G₄S | 44.9 | 1.59 | 43.31 | |
| SEQ124 | TNFRSF1B | IL9R | IL9R | QP | 52.2 | 1.11 | 51.09 | |
| SEQ153 + | IL10Ra | IL9R | IL9R | G₄S | 19.2 | 0.68 | | 18.52 |
| CAR+ | | | | | | | | |
| SEQ155 | TGFBR2 | IL9R | IL9R | G₄S | 6.22 | 0.45 | 5.77 | |
| SEQ156 | TGFBR2 | IL9R | IL9R | QP | 10.7 | 0.17 | 10.53 | |
| SEQ159 + | TIGIT | IL9R | IL9R | G₄S | 13.8 | 0.16 | | 13.64 |
| CAR+ | | | | | | | | |
| SEQ160 + | TIGIT | IL9R | IL9R | QP | 19.9 | 0.023 | | 19.877 |
| CAR+ | | | | | | | | |
| SEQ165 | 2B4 | IL9R | IL9R | G₄S | 30.4 | 28.5 | 1.9 | |
| SEQ166 | 2B4 | IL9R | IL9R | QP | 18.1 | 15.3 | 2.8 | |
| SEQ187 | DCR1 | TNFR1 | IL9R | G₄S | 35.3 | 15.5 | 19.8 | |
| SEQ188 | DCR1 | TNFR1 | IL9R | QP | 47 | 11.4 | 35.6 | |
| SEQ189 | CD40 | TNFR1 | IL9R | G₄S | 31.1 | 1.03 | 30.07 | |
| SEQ190 | CD40 | TNFR1 | IL9R | QP | 29 | 1.45 | 27.55 | |
| SEQ193 | DR4 | TNFR1 | IL9R | G₄S | 65.8 | 45.3 | 20.5 | |
| SEQ194 | DR4 | TNFR1 | IL9R | QP | 63.7 | 49.6 | 14.1 | |
| SEQ200 | TNFRSF1B | TNFR1 | IL9R | G₄S | 29.1 | 1.31 | 27.79 | |
| SEQ201 | TNFRSF1B | TNFR1 | IL9R | QP | 47.3 | 0.95 | 46.35 | |

As demonstrated by the data in Table 4, all constructs exhibited some level of cell surface expression in 293T cells. These results indicate that the constructs produced properly folded proteins capable of endocytic transport and resistant to general proteolysis.

Constructs that exhibited cell surface expression in 293T cells were then packaged in lentivirus and titered on human primary T cells. The expression analysis over transduced primary human T cells is show in Table 5 below

TABLE 5

| | | | | | | | | | % positive |
|---|---|---|---|---|---|---|---|---|---|
| | | | | | | | | % positive | T cells in |
| | | | | | | | % double- | T cells in | stained un- |
| | | | | | | | positive | unstained | transduced |
| | Ligand- | Trans- | Intra- | | Fold- | % | T cells | T cells | T cells |
| Sequence | binding | membrane | cellular | | dilution of | positive | (average of | (average of | (endogenous |
| ID | domain | domain | domain | Linker | lentivirus | T cells | duplicates) | duplicates) | expression) |
| SEQ63 + | IL21R | IL9R | IL9R | N/A | 2 | | 61 | 0.46 | 20.6 |
| CAR+ | | | | | 6 | | 39.85 | | |
| | | | | | 18 | | 28.4 | | |
| | | | | | 54 | | 14.8 | | |
| | | | | | 162 | | 4.725 | | |
| | | | | | 486 | | 0.845 | | |
| | | | | | 1458 | | 0.495 | | |
| SEQ66 | IL15Ra | IL9R | IL9R | N/A | 2 | 63.1 | | 0.2 | 32.3 |
| | | | | | 6 | 60.6 | | | |
| | | | | | 18 | 33.85 | | | |
| | | | | | 54 | 40.6 | | | |
| | | | | | 162 | 39.6 | | | |
| | | | | | 486 | 38 | | | |
| | | | | | 1458 | 36 | | | |
| SEQ69 | IL7Ra | IL9R | IL9R | N/A | 2 | 90.6 | | 1.02 | 55.5 |
| | | | | | 6 | 83.4 | | | |
| | | | | | 18 | 77.25 | | | |
| | | | | | 54 | 66.6 | | | |
| | | | | | 162 | 60.45 | | | |
| | | | | | 486 | 54.4 | | | |
| | | | | | 1458 | 52.8 | | | |
| SEQ72 + | IL4Ra | IL9R | IL9R | N/A | 2 | | 38.95 | 0.15 | 44.6 |
| CAR+ | | | | | 6 | | 29.85 | | |
| | | | | | 18 | | 18.1 | | |
| | | | | | 54 | | 10.8 | | |
| | | | | | 162 | | 7.01 | | |
| | | | | | 486 | | 2.915 | | |
| | | | | | 1458 | | 1.41 | | |
| SEQ89 + | CTLA4 | IL9R | IL9R | G₄S | 2 | | 30 | N/A | N/A |
| CAR+ | | | | | 6 | | 18.45 | | |
| | | | | | 18 | | 9.895 | | |
| | | | | | 54 | | 3.97 | | |
| | | | | | 162 | | 1.355 | | |
| | | | | | 486 | | 0.635 | | |
| | | | | | 1458 | | 0.485 | | |
| SEQ90 + | CTLA4 | IL9R | IL9R | QP | 2 | | 41.3 | 0.058 | 0.63 |
| CAR+ | | | | | 6 | | 31.5 | | |
| | | | | | 18 | | 20.05 | | |
| | | | | | 54 | | 12.6 | | |
| | | | | | 162 | | 3.06 | | |
| | | | | | 486 | | 1.255 | | |
| | | | | | 1458 | | 0.68 | | |
| SEQ91 | TIM3 | IL9R | IL9R | G₄S | 2 | 83.4 | | 1.71 | 88.4 |
| | | | | | 6 | 84.15 | | | |
| | | | | | 18 | 85.5 | | | |
| | | | | | 54 | 81.8 | | | |
| | | | | | 162 | 88.3 | | | |
| | | | | | 486 | 86.6 | | | |
| | | | | | 1458 | 77 | | | |
| | | | | | 4374 | 84.9 | | | |
| SEQ95 + | PD1 | IL9R | IL9R | G₄S | 2 | | 32.45 | 0.027 | 0.64 |
| CAR+ | | | | | 6 | | 18.75 | | |
| | | | | | 18 | | 14.45 | | |
| | | | | | 54 | | 6.965 | | |
| | | | | | 162 | | 2.43 | | |
| | | | | | 486 | | 1.19 | | |
| | | | | | 1458 | | 0.88 | | |
| SEQ96 + | PD1 | IL9R | IL9R | QP | 2 | | 30.5 | 0.01 | 85.8 |
| CAR+ | | | | | 6 | | 25.1 | | |
| | | | | | 18 | | 12.85 | | |
| | | | | | 54 | | 9.4 | | |
| | | | | | 162 | | 3.31 | | |
| | | | | | 486 | | 1.33 | | |
| | | | | | 1458 | | 0.645 | | |
| SEQ109 | DCR1 | IL9R | IL9R | G₄S | 2 | 77.8 | | 0.033 | 0.7 |
| | | | | | 6 | 60.95 | | | |
| | | | | | 18 | 44.6 | | | |
| | | | | | 54 | 29.45 | | | |
| | | | | | 162 | 13.4 | | | |
| | | | | | 486 | 4.285 | | | |
| | | | | | 1458 | 2.985 | | | |
| SEQ110 | DCR1 | IL9R | IL9R | QP | 2 | 89.1 | | 0.059 | 1.14 |
| | | | | | 6 | 74.9 | | | |
| | | | | | 18 | 49.65 | | | |
| | | | | | 54 | 28.9 | | | |
| | | | | | 162 | 15.6 | | | |
| | | | | | 486 | 10.755 | | | |
| | | | | | 1458 | 8.61 | | | |
| SEQ111 | CD40 | IL9R | IL9R | G₄S | 2 | 50.25 | | 0.44 | 12.1 |
| | | | | | 6 | 48.2 | | | |
| | | | | | 18 | 38.1 | | | |
| | | | | | 54 | 40.25 | | | |
| | | | | | 162 | 21.8 | | | |
| | | | | | 486 | 9.285 | | | |
| | | | | | 1458 | 9.3 | | | |
| SEQ112 | CD40 | IL9R | IL9R | QP | 2 | 85.05 | | 0.049 | 3.41 |
| | | | | | 6 | 78.2 | | | |
| | | | | | 18 | 62.4 | | | |
| | | | | | 54 | 39.1 | | | |
| | | | | | 162 | 31.75 | | | |
| | | | | | 486 | 13.65 | | | |
| | | | | | 1458 | 12.13 | | | |
| SEQ113 + | Fas | IL9R | IL9R | G₄S | 2 | | 42.45 | N/A | N/A |
| CAR+ | | | | | 6 | | 17.3 | | |
| | | | | | 18 | | 8.735 | | |
| | | | | | 54 | | 4.84 | | |
| | | | | | 162 | | 2.05 | | |
| | | | | | 486 | | 0.98 | | |
| | | | | | 1458 | | 0.625 | | |
| SEQ114 + | Fas | IL9R | IL9R | QP | 2 | | 87.15 | 0.34 | 98.1 |
| CAR+ | | | | | 6 | | 81.25 | | |
| | | | | | 18 | | 71.4 | | |
| | | | | | 54 | | 54.15 | | |
| | | | | | 162 | | 30 | | |
| | | | | | 486 | | 13 | | |
| | | | | | 1458 | | 4.52 | | |
| SEQ116 | DR4 | IL9R | IL9R | QP | 2 | 93.1 | | 0.2 | 33.9 |
| | | | | | 6 | 87.85 | | | |
| | | | | | 18 | 77.75 | | | |
| | | | | | 54 | 63.8 | | | |
| | | | | | 162 | 50.1 | | | |
| | | | | | 486 | 39 | | | |
| | | | | | 1458 | 34.6 | | | |
| SEQ123 | TNFRSF1B | IL9R | IL9R | G₄S | 2 | 90.9 | | 0.019 | 68.5 |
| | | | | | 6 | 86.45 | | | |
| | | | | | 18 | 77.85 | | | |
| | | | | | 54 | 57.75 | | | |
| | | | | | 162 | 78 | | | |
| | | | | | 486 | 72.45 | | | |
| | | | | | 1458 | 72.5 | | | |
| SEQ124 | TNFRSF1B | IL9R | IL9R | QP | 2 | 81.05 | | 0.086 | 72.7 |
| | | | | | 6 | 78.25 | | | |
| | | | | | 18 | 70 | | | |
| | | | | | 54 | 70.75 | | | |
| | | | | | 162 | 71.9 | | | |
| | | | | | 486 | 73.6 | | | |
| | | | | | 1458 | 71.65 | | | |
| SEQ154 + | IL10Ra | IL9R | IL9R | QP | 2 | | 28.2 | 0.33 | 1.34 |
| CAR+ | | | | | 6 | | 22.4 | | |
| | | | | | 18 | | 10.9 | | |
| | | | | | 54 | | 4.49 | | |
| | | | | | 162 | | 1.635 | | |
| | | | | | 486 | | 1.055 | | |
| | | | | | 1458 | | 1.075 | | |
| SEQ155 | TGFBR2 | IL9R | IL9R | G₄S | 2 | 27.55 | | 0.67 | 0.68 |
| | | | | | 6 | 17.6 | | | |
| | | | | | 18 | 14.14 | | | |
| | | | | | 54 | 4.305 | | | |
| | | | | | 162 | 1.425 | | | |
| | | | | | 486 | 2.1 | | | |
| | | | | | 1458 | 1.6 | | | |
| SEQ156 | TGFBR2 | IL9R | IL9R | QP | 2 | 77.45 | | 0.056 | 68.9 |
| | | | | | 6 | 76.8 | | | |
| | | | | | 18 | 70.55 | | | |
| | | | | | 54 | 70.9 | | | |
| | | | | | 162 | 63.45 | | | |
| | | | | | 486 | 67.5 | | | |
| | | | | | 1458 | 71.05 | | | |
| SEQ171 | TWEAKR | IL9R | IL9R | G₄S | 2 | 77.35 | | 0.54 | 62.8 |
| | | | | | 6 | 68.45 | | | |
| | | | | | 18 | 53.65 | | | |
| | | | | | 54 | 46.8 | | | |
| | | | | | 162 | 59.7 | | | |
| | | | | | 486 | 63.4 | | | |
| | | | | | 1458 | 63.75 | | | |
| SEQ172 | TWEAKR | IL9R | IL9R | QP | 2 | 74 | | 0.39 | 32.9 |
| | | | | | 6 | 53.85 | | | |
| | | | | | 18 | 44.9 | | | |
| | | | | | 54 | 47.4 | | | |
| | | | | | 162 | 36.45 | | | |
| | | | | | 486 | 34.75 | | | |
| | | | | | 1458 | 34.1 | | | |
| SEQ173 | TWEAKR | TNFR1 | IL9R | G₄S | 2 | 92.7 | | 0.37 | 30.5 |
| | | | | | 6 | 80.85 | | | |
| | | | | | 18 | 64.65 | | | |
| | | | | | 54 | 46.55 | | | |
| | | | | | 162 | 36.1 | | | |
| | | | | | 486 | 32.95 | | | |
| | | | | | 1458 | 31 | | | |
| SEQ174 | TWEAKR | TNFR1 | IL9R | QP | 2 | 87.2 | | 0.29 | 23.6 |
| | | | | | 6 | 68.95 | | | |
| | | | | | 18 | 47.75 | | | |
| | | | | | 54 | 37.15 | | | |
| | | | | | 162 | 30 | | | |
| | | | | | 486 | 25.7 | | | |
| | | | | | 1458 | 26.75 | | | |
| SEQ187 | DCR1 | TNFR1 | IL9R | G₄S | 2 | 86.65 | | 0.16 | 4.81 |
| | | | | | 6 | 70.25 | | | |
| | | | | | 18 | 50.15 | | | |
| | | | | | 54 | 29 | | | |
| | | | | | 162 | 19.8 | | | |
| | | | | | 486 | 6.475 | | | |
| | | | | | 1458 | 3.325 | | | |
| SEQ188 | DCR1 | TNFR1 | IL9R | QP | 2 | 63.85 | | 0.02 | 8.63 |
| | | | | | 6 | 60.8 | | | |
| | | | | | 18 | 48.65 | | | |
| | | | | | 54 | 35.7 | | | |
| | | | | | 162 | 15.08 | | | |
| | | | | | 486 | 14.7 | | | |
| | | | | | 1458 | 8.65 | | | |
| SEQ189 | CD40 | TNFR1 | IL9R | G₄S | 2 | 91.05 | | 0.079 | 11.9 |
| | | | | | 6 | 79 | | | |
| | | | | | 18 | 55.8 | | | |
| | | | | | 54 | 24.3 | | | |
| | | | | | 162 | 20.55 | | | |
| | | | | | 486 | 10.65 | | | |
| | | | | | 1458 | 10.365 | | | |
| SEQ190 | CD40 | TNFR1 | IL9R | QP | 2 | 58.15 | | 0.32 | 34 |
| | | | | | 6 | 56.3 | | | |
| | | | | | 18 | 53.5 | | | |
| | | | | | 54 | 48.5 | | | |
| | | | | | 162 | 38.65 | | | |
| | | | | | 486 | 33.15 | | | |
| | | | | | 1458 | 31.3 | | | |
| SEQ193 | DR4 | TNFR1 | IL9R | G₄S | 2 | 86 | | 0.22 | 40 |
| | | | | | 6 | 69.3 | | | |
| | | | | | 18 | 61 | | | |
| | | | | | 54 | 50.3 | | | |
| | | | | | 162 | 48.85 | | | |
| | | | | | 486 | 47 | | | |
| | | | | | 1458 | 43.2 | | | |
| SEQ194 | DR4 | TNFR1 | IL9R | QP | 2 | 87 | | 0.089 | 60.6 |
| | | | | | 6 | 84.3 | | | |
| | | | | | 18 | 83.4 | | | |
| | | | | | 54 | 84.15 | | | |
| | | | | | 162 | 75.6 | | | |
| | | | | | 486 | 74 | | | |
| | | | | | 1458 | 64.85 | | | |
| SEQ200 | TNFRSF1B | TNFR1 | IL9R | G₄S | 2 | 96.95 | | 1.84 | 93.6 |
| | | | | | 6 | 95.35 | | | |
| | | | | | 18 | 93.65 | | | |
| | | | | | 54 | 94.25 | | | |
| | | | | | 162 | 92.7 | | | |
| | | | | | 486 | 91.65 | | | |
| | | | | | 1458 | 92 | | | |
| SEQ201 | TNFRSF1B | TNFR1 | IL9R | QP | 2 | 95.85 | | 0.39 | 92.8 |
| | | | | | 6 | 96.2 | | | |
| | | | | | 18 | 94.15 | | | |
| | | | | | 54 | 93.5 | | | |
| | | | | | 162 | 94.15 | | | |
| | | | | | 486 | 92.1 | | | |
| | | | | | 1458 | 93.8 | | | |

As demonstrated in Table 5 above, lentiviruses for all constructs were successfully produced and transduced primary human T cells exhibited at least some level of cell surface expression. Despite their general resistance to viral transduction and sensitivity unfolded protein responses (UPR) [Lopez-Soto et al., “Cancer-induced Endoplasmic Reticulum Stress in T Cells Subverts Immunosurveillance,” Cell Metabolism 28 (6): 803-805 (2018); Li et al., “The Emerging Roles of Endoplasmic Reticulum Stress in Balancing Immunity and Tolerance in Health and Diseases: Mechanisms and Opportunities,” Front. Immunol. Volum 10, Article 3154], the constructs produced properly folded proteins capable of endocytic transport and resistant to general proteolysis in primary T cells.

### Example 10

### Expression of Chimeric Switch Receptors Results in Phosphoryation of Stat Receptors

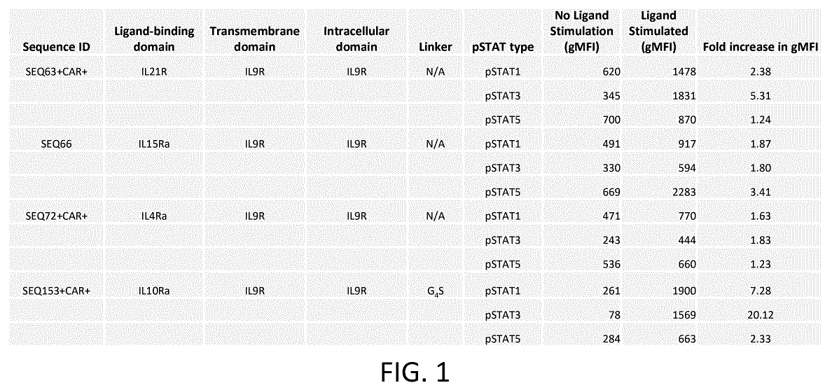

Some constructs were then tested for their ability to induce phosphorylation of STAT1, STAT3, and/or STAT5. Briefly, primary human T cells were transduced with a lentiviral vector encoding switch receptors of SEQ ID NO:63+CAR+(IL21R ECD+IL9R TM+IL9R ICD and CAR 4D5), SEQ ID NO:66 (IL15Ra+IL9R TM+IL9R ICD), SEQ ID NO:72+CAR+(ILAR ECD+IL9R TM +IL9R ICD and CAR 4D5), or SEQ ID NO:153+CAR+(IL10Ra ECD+IL9R TM+IL9R ICD and CAR 4D5). Cells transduced with SEQ ID NO:63+CAR+ were left either unstimulated or stimulated with 200 ng/ml IL-21 for 30 minutes. Cells transduced with SEQ ID NO: 66 were left either unstimulated or stimulated with 200 ng/mL IL-15 for 30 minutes. Cells transduced with SEQ ID NO:72+CAR+ were left either unstimulated or stimulated with 200 ng/ml IL-4 for 30 minutes. Cells transduced with SEQ ID NO:153+CAR+ were left either unstimulated or stimulated with 200 ng/ml IL-4 for 30 minutes. The fold increase in gMFI of ligand stimulated versus no stimulation was calculated and is shown in FIG. 1.

As shown in FIG. 1, all constructs induced phosphorylation of STAT1, STAT3, and STAT5 when stimulated with ligand. Activation of STAT family members through ligand mediated phosphorylation is believed to confer advantages in effector function, polarization and proliferation to T cells.

### Example 11

### Primary T Cells Expressing Chimeric Switch Receptors Exhibit Enhanced Tumor Cell Killing

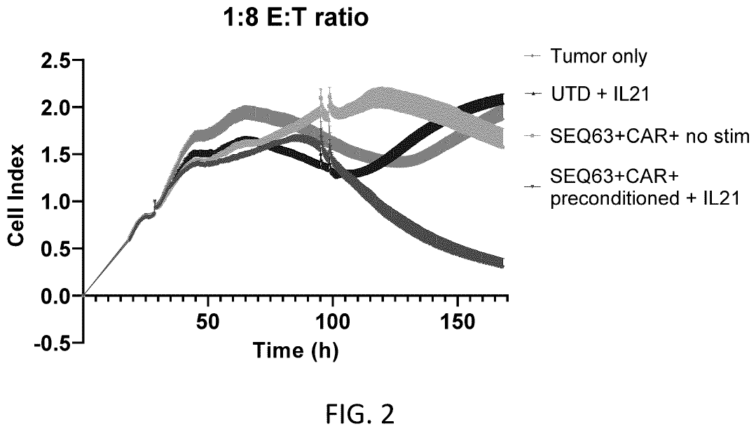

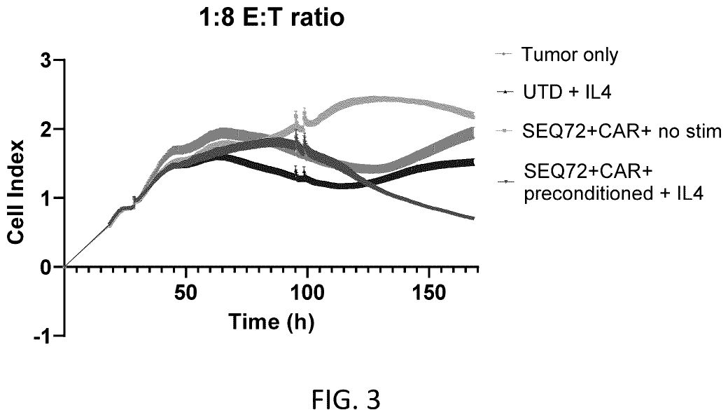

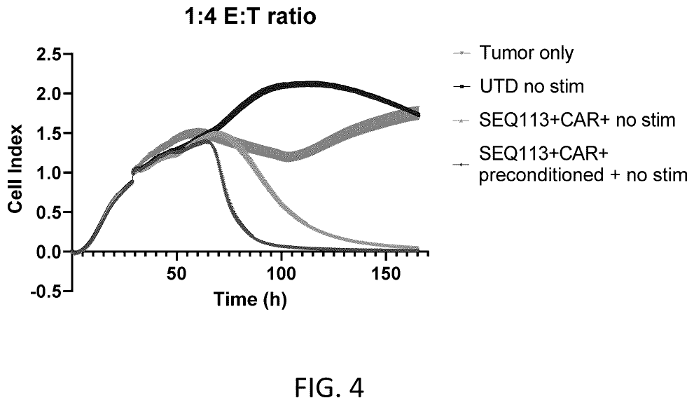

Real-time cytotoxicity assays were then used to analyze whether or not cells expressing a chimeric switch receptor of the present disclosure would show increased cytotoxicity when exposed to ligand. As shown in FIGS. 2-4, upon stimulation with ligand, cells expressing either SEQ ID NO:63+CAR+(IL21R ECD+IL9R TM+IL9R ICD and CAR 4D5) (FIG. 2), SEQ ID NO: 72+CAR+(ILAR ECD+IL9R TM+IL9R ICD and CAR 4D5) (FIG. 3), or SEQ ID NO: 113+CAR+(Fas ECD+IL9R TM+IL9R ICD and CAR 4D5) (FIG. 4), demonstrated an increase in killing of SKOV-3 human ovarian adenocarcinoma cells expressing HER2 when preconditioned with ligand as compared to untransduced, stimulated cells. Consistent with the observed activation of STAT transcription factors following ligand stimulation, T cells transduced with hybrid IL9R receptors demonstrated improved killing over unstimulated controls. It should be noted that in the case of Seq ID NO 113, the presence of natural ligand (TNFSF6) increases background killing in the assay. That said, the addition of exogenous ligand enhances T cell killing above controls.

### Example 12

### Primary T Cells Expressing Chimeric Switch Receptors Exhibit Increased Cytokine and Chemokine Activity

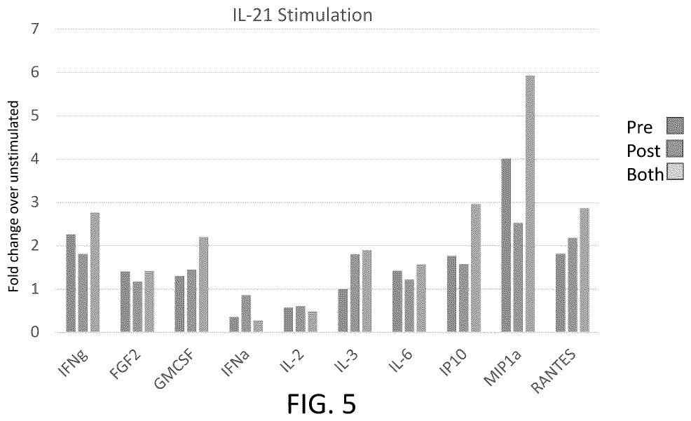

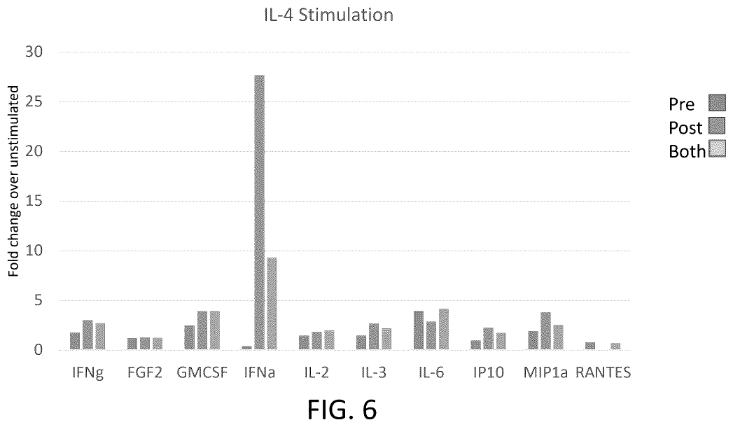

Culture supernatants from primary human T cells transduced with either SEQ ID NO: 63+CAR+(IL21R ECD+IL9R TM+IL9R ICD and CAR 4D5) (FIG. 5) or SEQ ID NO: 72+CAR+(ILAR ECD+IL9R TM+IL9R ICD and CAR 4D5) (FIG. 6) were then analyzed for the presence of cytokines and chemokines. Supernatants were taken from cells stimulated with ligand before the real-time cytotoxicity assay of Example 11 (first bar, “pre”), cells stimulated with ligand during the real-time cytotoxicity assay of Example 11 (second bar, “post”), or cells stimulated at both points in the real-time cytotoxicity assay of Example 11 (third bar, “both”). The fold increase over unstimulated was calculated and is shown in FIGS. 5 and 6. As shown in FIGS. 5 and 6, several cytokines and chemokines, such as IFNg, FGF2, GMCSF, IL-3, IL-6, IP10, MIP1a, and RANTES, demonstrated increased production. The magnitude and quality of cytokines produced following ligand stimulation (either “pre”, “post” or “both”) indicates a robust improvement in T cell response with hybrid-IL9R engagement. Furthermore, the pattern of cytokine response indicates an increase in type-1 polarization following ligand stimulation relative to controls.

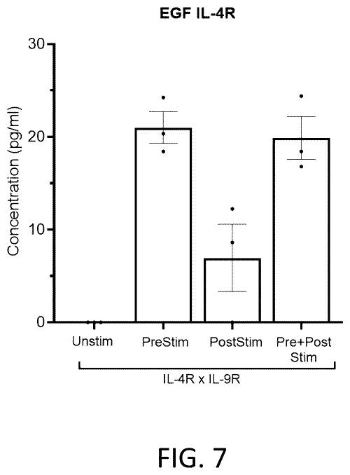

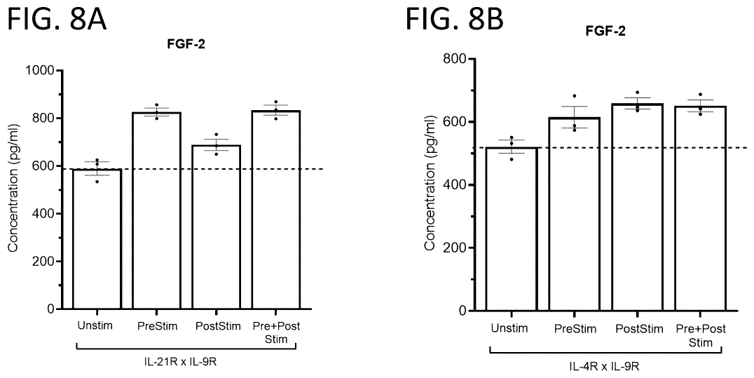

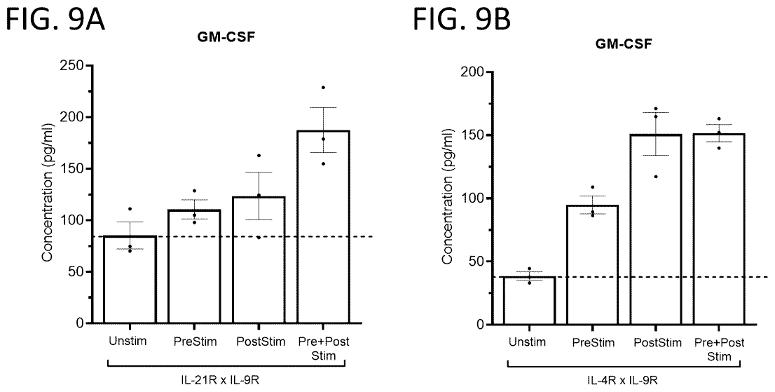

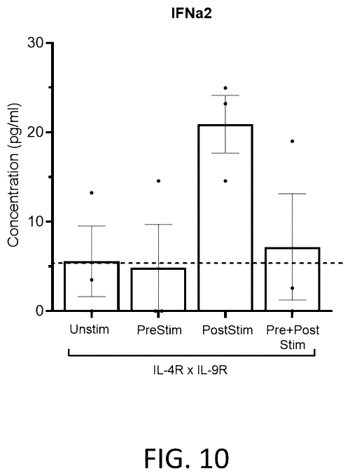

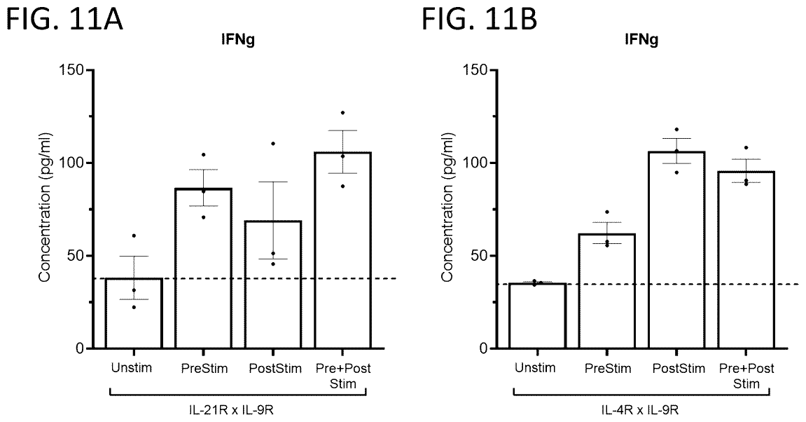

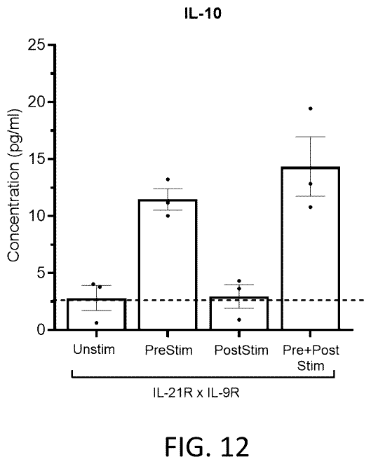

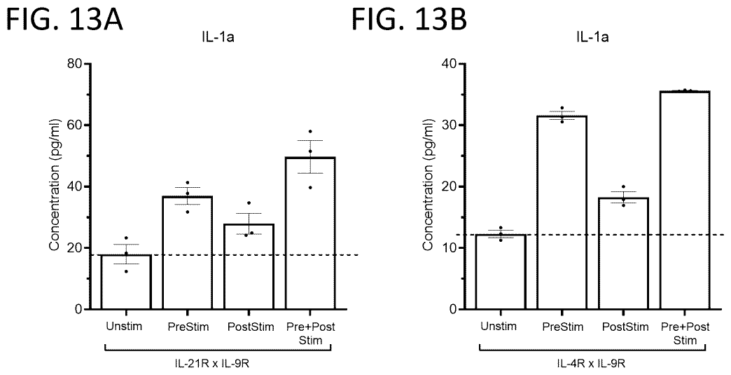

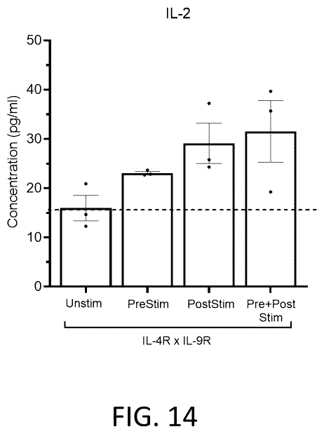

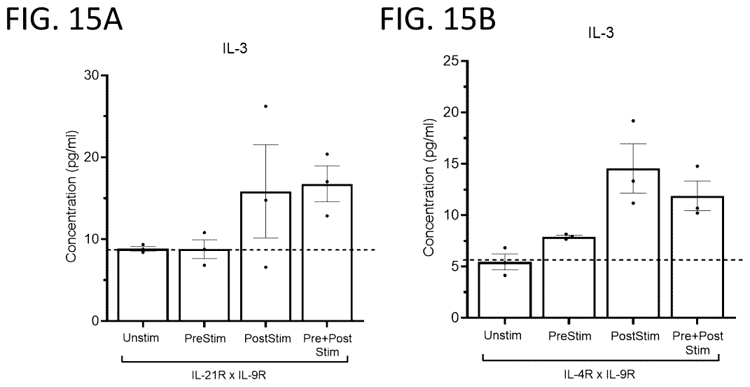

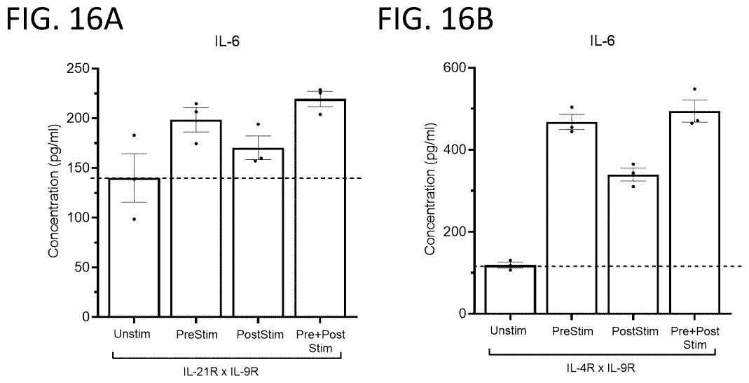

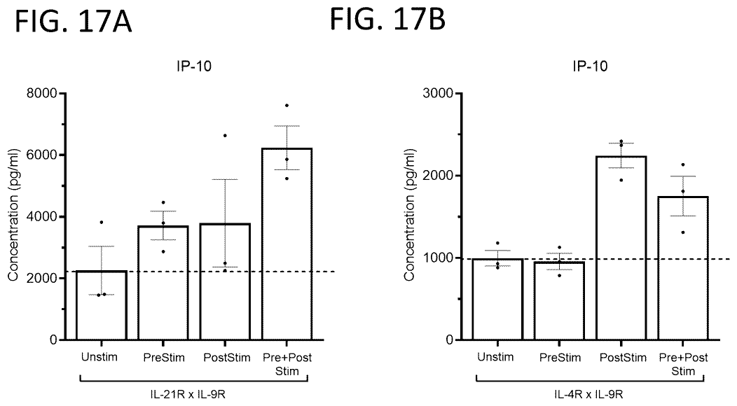

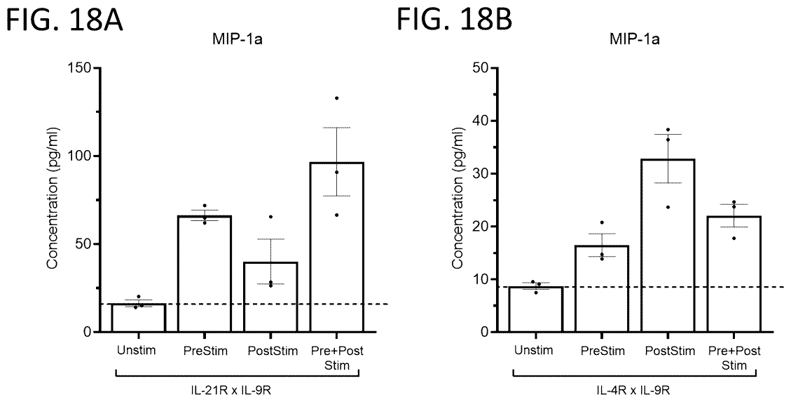

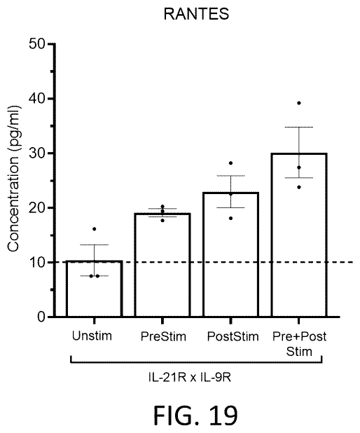

FIGS. 7-19 depict results from the same experiment, however, the data is shown in concentration of cytokine or chemokine (in pg.mL) for each of the above described experimental conditions. Similarly, the magnitude and quality of cytokines produced following ligand stimulation (either “pre”, “post” or “both”) indicates a robust improvement in T cell response with hybrid-IL9R engagement, and the pattern of cytokine response indicates an increase in type-1 polarization following ligand stimulation relative to controls.

What is claimed is:

1. A chimeric receptor polypeptide comprising:
an extracellular portion comprising a binding domain of an endogenous cytokine receptor;
an intracellular portion comprising an endodomain of an IL-9 receptor; and
a transmembrane domain that joins the extracellular portion and the intracellular portion.

2. The polypeptide of claim 1, further comprising one or more linkers.

3. The polypeptide of claim 1, wherein the endogenous cytokine receptor is selected from IL-2rb, IL-2ra, IL-4r, IL-7ra, IL-15ra and IL-21ra.

4. The polypeptide of claim 1, wherein the polypeptide comprises an amino acid sequence having at least 80% sequence identity to an amino acid sequence selected from SEQ ID Nos: 1-6.

5. The polypeptide of claim 1, wherein the transmembrane domain is selected from the transmembrane domain of IL-9, IL-7ra, IL-2rb and TNFR1.

6. The polypeptide of claim 1, wherein the polypeptide comprises an amino acid sequence selected from SEQ ID Nos: 53-56.

7. The polypeptide of claim 1, wherein the polypeptide comprises an amino acid sequence having at least 80% sequence identity to an amino acid sequence selected from SEQ ID Nos: 63-80.

8. A cell comprising the polypeptide of claim 1.

9. The cell of claim 8, wherein the cell is a eukaryotic cell.

10. The cell of claim 9, wherein the eukaryotic cell is an animal cell.

11. The cell of claim 10, wherein the animal cell is a mammalian cell.

12. The cell of claim 11, wherein the mammalian cell is an immune cell, a neuron, an epithelial cell, an endothelial cell or a stem cell.

13. The cell of claim 12, wherein the cell is an immune cell or a dendritic cell.

14. The cell of claim 12, wherein the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}) or other T cell.


SEQUENCE LISTING (205 sequences)

SEQ ID NO: 1 - protein, 214 aa, synthetic construct
  REGION 1..214: note = MISC_FEATURE - IL-2rb
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 2 - protein, 219 aa, synthetic construct
  REGION 1..219: note = MISC_FEATURE - IL-2ra
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 3 - protein, 207 aa, synthetic construct
  REGION 1..207: note = MISC_FEATURE - IL-4
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 4 - protein, 219 aa, synthetic construct
  REGION 1..219: note = MISC_FEATURE - IL-7ra
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 5 - protein, 175 aa, synthetic construct
  REGION 1..175: note = MISC_FEATURE - IL-15ra
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 6 - protein, 213 aa, synthetic construct
  REGION 1..213: note = MISC_FEATURE - IL-21ra
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 7 - protein, 93 aa, synthetic construct
  REGION 1..93: note = MISC_FEATURE - TGFBR1
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 8 - protein, 144 aa, synthetic construct
  REGION 1..144: note = MISC_FEATURE - TGFBR2
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 9 - protein, 214 aa, synthetic construct
  REGION 1..214: note = MISC_FEATURE - IL-10ra
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 10 - protein, 148 aa, synthetic construct
  REGION 1..148: note = MISC_FEATURE - FAS
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 11 - protein, 126 aa, synthetic construct [sequence omitted]

SEQ ID NO: 12 - protein, 428 aa, synthetic construct
  REGION 1..428: note = MISC_FEATURE - LAG3
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 13 - protein, 181 aa, synthetic construct
  REGION 1..181: note = MISC_FEATURE - TIM3
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 14 - protein, 220 aa, synthetic construct
  REGION 1..220: note = MISC_FEATURE - PD1
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 15 - protein, 438 aa, synthetic construct
  REGION 1..438: note = MISC_FEATURE - ILT2
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 16 - protein, 238 aa, synthetic construct
  REGION 1..238: note = MISC_FEATURE - ILT3
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE

SEQ ID NO: 17 - protein, 440 aa, synthetic construct
  REGION 1..440: note = MISC_FEATURE - ILT4
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGV

SEQ ID NO: 18 - protein, 420 aa, synthetic construct
  REGION 1..420: note = MISC_FEATURE - ILT5
GPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLE

SEQ ID NO: 19 - protein, 732 aa, synthetic construct
  REGION 1..732: note = MISC_FEATURE - VEGFR1
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLE

SEQ ID NO: 20 - protein, 745 aa, synthetic construct
  REGION 1..745: note = MISC_FEATURE - VEGFR2
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 21 - protein, 751 aa, synthetic construct
  REGION 1..751: note = MISC_FEATURE - VEGFR3
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSME

SEQ ID NO: 22 - protein, 380 aa, synthetic construct
  REGION 1..380: note = MISC_FEATURE - OPG
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 23 - protein, 165 aa, synthetic construct
  REGION 1..165: note = MISC_FEATURE - TACI
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYS

SEQ ID NO: 24 - protein, 54 aa, synthetic construct
  REGION 1..54: note = MISC_FEATURE - BCM4
MLQMAGQCSQNEYFDSLLHACIPCQLRCSSNTPPLTCQRYCNASVTNSVKGTNA

SEQ ID NO: 25 - protein, 222 aa, synthetic construct
  REGION 1..222: note = MISC_FEATURE - NGFR
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDN

SEQ ID NO: 26 - protein, 161 aa, synthetic construct
  REGION 1..161: note = MISC_FEATURE - EDAR
EYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATA

SEQ ID NO: 27 - protein, 156 aa, synthetic construct
  REGION 1..156: note = MISC_FEATURE - DCR2 (TNFRSF10D)
ATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYH

SEQ ID NO: 28 - protein, 211 aa, synthetic construct
  REGION 1..211: note = MISC_FEATURE - DCR1 (TNFRSF10C)
ATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMITSPGTPA

SEQ ID NO: 29 - protein, 173 aa, synthetic construct
  REGION 1..173: note = MISC_FEATURE - CD40
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 30 - protein, 216 aa, synthetic construct
  REGION 1..216: note = MISC_FEATURE - DR4
ASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHN

SEQ ID NO: 31 - protein, 308 aa, synthetic construct
  REGION 1..308: note = MISC_FEATURE - DR6
QPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEH

SEQ ID NO: 32 - protein, 155 aa, synthetic construct
  REGION 1..155: note = MISC_FEATURE - DR5
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCS

SEQ ID NO: 33 - protein, 175 aa, synthetic construct
  REGION 1..175: note = MISC_FEATURE - DR3
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 34 - protein, 235 aa, synthetic construct
  REGION 1..235: note = MISC_FEATURE - TNFRSF1B
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 35 - protein, 182 aa, synthetic construct
  REGION 1..182: note = MISC_FEATURE - TNFRSF1
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 36 - protein, 113 aa, synthetic construct
  REGION 1..113: note = MISC_FEATURE - BMPR1B
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 37 - protein, 129 aa, synthetic construct
  REGION 1..129: note = MISC_FEATURE - BMPR1A
QNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIR

SEQ ID NO: 38 - protein, 124 aa, synthetic construct [sequence omitted]

SEQ ID NO: 39 - protein, 603 aa, synthetic construct
  REGION 1..603: note = MISC_FEATURE - CSF3R
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELH

SEQ ID NO: 40 - protein, 498 aa, synthetic construct
  REGION 1..498: note = MISC_FEATURE - CSF1R
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTP

SEQ ID NO: 41 - protein, 103 aa, synthetic construct
  REGION 1..103: note = MISC_FEATURE - Activin R1A
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLE

SEQ ID NO: 42 - protein, 103 aa, synthetic construct
  REGION 1..103: note = MISC_FEATURE - Activin R1B
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVE

SEQ ID NO: 43 - protein, 92 aa, synthetic construct
  REGION 1..92: note = MISC_FEATURE - Activin R1C
LSPGLKCVCLLCDSSNFTCQTEGACWASVMLTNGKEQVIKSCVSLPELNAQVFCHSSNNVTKTECCFTDFCNNITLHLPTASPNAPKLGPME

SEQ ID NO: 44 - protein, 119 aa, synthetic construct
  REGION 1..119: note = MISC_FEATURE - Activin R2B
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLT

SEQ ID NO: 45 - protein, 116 aa, synthetic construct
  REGION 1..116: note = MISC_FEATURE - Activin R2A
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 46 - protein, 120 aa, synthetic construct
  REGION 1..120: note = MISC_FEATURE - TIGIT
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 47 - protein, 175 aa, synthetic construct
  REGION 1..175: note = MISC_FEATURE - FCGR2B
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAP

SEQ ID NO: 48 - protein, 277 aa, synthetic construct [sequence omitted]

SEQ ID NO: 49 - protein, 208 aa, synthetic construct
  REGION 1..208: note = MISC_FEATURE - 2B4
CQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWP

SEQ ID NO: 50 - protein, 144 aa, synthetic construct
  REGION 1..144: note = MISC_FEATURE - LAIR1
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLY

SEQ ID NO: 51 - protein, 348 aa, synthetic construct
  REGION 1..348: note = MISC_FEATURE - CD5
RLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNP

SEQ ID NO: 52 - protein, 53 aa, synthetic construct
  REGION 1..53: note = MISC_FEATURE - TWEAKR
EQAPGTAPCSRGSSWSADLDKCMDCASCRARPHSDFCLGCAAAPPAPFRLLWP

SEQ ID NO: 53 - protein, 34 aa, synthetic construct
  REGION 1..34: note = MISC_FEATURE - IL-9
LIPPWGWPGNTLVAVSIFLLLTGPTYLLFKLSPR

SEQ ID NO: 54 - protein, 25 aa, synthetic construct
  REGION 1..25: note = MISC_FEATURE - IL-7ra
PILLTISILSFFSVALLVILACVLW

SEQ ID NO: 55 - protein, 25 aa, synthetic construct
  REGION 1..25: note = MISC_FEATURE - IL-2rb
IPWLGHLLVGLSGAFGFIILVYLLI

SEQ ID NO: 56 - protein, 23 aa, synthetic construct
  REGION 1..23: note = MISC_FEATURE - TNFR1
VLLPLVIFFGLCLLSLLFIGLMY

SEQ ID NO: 57 - protein, 225 aa, synthetic construct
  REGION 1..225: note = MISC_FEATURE - IL-9
VKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 58 - protein, 86 aa, synthetic construct
  REGION 1..86: note = MISC_FEATURE - BOX1/2
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 59 - protein, 15 aa, synthetic construct
  REGION 1..15: note = MISC_FEATURE - Gly Ser Linker
GGGGSGGGGSGGGGS

SEQ ID NO: 60 - protein, 12 aa, synthetic construct
  REGION 1..12: note = MISC_FEATURE - Q-Pro Linker
QPQPQPQPQPQP

SEQ ID NO: 61 - protein, 12 aa, synthetic construct
  REGION 1..12: note = MISC_FEATURE - K-Pro Linker
KPKPKPKPKPKP

SEQ ID NO: 62 - protein, 26 aa, synthetic construct
  REGION 1..26: note = MISC_FEATURE - Exemplary signal sequence
MAAPALSWRLPLLILLLPLATSWASA

SEQ ID NO: 63 - protein, 472 aa, synthetic construct
  REGION 1..472: note = MISC_FEATURE - IL21r/IL9R
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 64 - protein, 463 aa, synthetic construct
  REGION 1..463: note = MISC_FEATURE - IL21r/IL9R/IL7RaTM
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEPILLTISILSFFSVALLVILACVLWVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 65 - protein, 463 aa, synthetic construct
  REGION 1..463: note = MISC_FEATURE - IL21r/IL9R/IL2RbTM
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEIPWLGHLLVGLSGAFGFIILVYLLIVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 66 - protein, 434 aa, synthetic construct
  REGION 1..434: note = MISC_FEATURE - IL15r-alpha/IL9R
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 67 - protein, 425 aa, synthetic construct
  REGION 1..425: note = MISC_FEATURE - IL15r-alpha/IL9R/IL7RaTM
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTPILLTISILSFFSVALLVILACVLWVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 68 - protein, 425 aa, synthetic construct
  REGION 1..425: note = MISC_FEATURE - IL15r-alpha/IL9R/IL2RbTM
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTIPWLGHLLVGLSGAFGFIILVYLLIVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 69 - protein, 478 aa, synthetic construct
  REGION 1..478: note = MISC_FEATURE - IL7r-alpha /IL9R
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 70 - protein, 469 aa, synthetic construct
  REGION 1..469: note = MISC_FEATURE - IL7r-alpha /IL9R/IL7RaTM
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 71 - protein, 469 aa, synthetic construct
  REGION 1..469: note = MISC_FEATURE - IL7r-alpha /IL9R/IL2RbTM
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDIPWLGHLLVGLSGAFGFIILVYLLIVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 72 - protein, 466 aa, synthetic construct
  REGION 1..466: note = MISC_FEATURE - IL4r-alpha/IL9R
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 73 - protein, 457 aa, synthetic construct
  REGION 1..457: note = MISC_FEATURE - IL4r-alpha/IL9R/IL7RaTM
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHPILLTISILSFFSVALLVILACVLWVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 74 - protein, 457 aa, synthetic construct
  REGION 1..457: note = MISC_FEATURE - IL4r-alpha/IL9R/IL2RbTM
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHIPWLGHLLVGLSGAFGFIILVYLLIVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 75 - protein, 473 aa, synthetic construct
  REGION 1..473: note = MISC_FEATURE - IL2r-beta Ectodomain /IL9R
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 76 - protein, 464 aa, synthetic construct
  REGION 1..464: note = MISC_FEATURE - IL2r-beta Ectodomain /IL9R/IL7RaTM
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTPILLTISILSFFSVALLVILACVLWVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 77 - protein, 464 aa, synthetic construct
  REGION 1..464: note = MISC_FEATURE - IL2r-beta Ectodomain /IL9R/IL2RbTM
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLIVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 78 - protein, 478 aa, synthetic construct
  REGION 1..478: note = MISC_FEATURE - IL2r-alpha Ectodomain/IL9R
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 79 - protein, 469 aa, synthetic construct
  REGION 1..469: note = MISC_FEATURE - IL2r-alpha Ectodomain/IL9R/IL7RaTM
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQPILLTISILSFFSVALLVILACVLWVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 80 - protein, 469 aa, synthetic construct
  REGION 1..469: note = MISC_FEATURE - IL2r-alpha Ectodomain/IL9R/IL2RbTM
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQIPWLGHLLVGLSGAFGFIILVYLLIVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 81 - protein, 780 aa, synthetic construct
  REGION 1..780: note = MISC_FEATURE - ILT5/IL9R/cGC-F
GPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 82 - protein, 777 aa, synthetic construct
  REGION 1..777: note = MISC_FEATURE - ILT5/IL9R/cGC-S
GPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 83 - protein, 598 aa, synthetic construct
  REGION 1..598: note = MISC_FEATURE - ILT3/IL9R/cGC-F
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 84 - protein, 595 aa, synthetic construct
  REGION 1..595: note = MISC_FEATURE - ILT3/IL9R/cGC-S
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 85 - protein, 800 aa, synthetic construct
  REGION 1..800: note = MISC_FEATURE - ILT4 IL9R/cGC-F
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 86 - protein, 797 aa, synthetic construct
  REGION 1..797: note = MISC_FEATURE - ILT4 IL9R/cGC-S
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 87 - protein, 798 aa, synthetic construct
  REGION 1..798: note = MISC_FEATURE - ILT2 IL9R/cGC-F
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 88 - protein, 795 aa, synthetic construct
  REGION 1..795: note = MISC_FEATURE - ILT2 IL9R/cGC-S
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 89 - protein, 486 aa, synthetic construct
  REGION 1..486: note = MISC_FEATURE - CTLA4 IL9R/cGC-F
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 90 - protein, 483 aa, synthetic construct
  REGION 1..483: note = MISC_FEATURE - CTLA4 IL9R/cGC-S
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 91 - protein, 541 aa, synthetic construct
  REGION 1..541: note = MISC_FEATURE - TIM3/IL9R/cGC-F
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 92 - protein, 538 aa, synthetic construct
  REGION 1..538: note = MISC_FEATURE - TIM3/IL9R/cGC-S
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 93 - protein, 785 aa, synthetic construct
  REGION 1..785: note = MISC_FEATURE - LAG3/IL9R/cGC-S
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 94 - protein, 788 aa, synthetic construct
  REGION 1..788: note = MISC_FEATURE - LAG3/IL9R/cGC-F
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 95 - protein, 580 aa, synthetic construct
  REGION 1..580: note = MISC_FEATURE - PD1/IL9R/cGC-F
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 96 - protein, 577 aa, synthetic construct
  REGION 1..577: note = MISC_FEATURE - PD1/IL9R/cGC-S
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 97 - protein, 740 aa, synthetic construct
  REGION 1..740: note = MISC_FEATURE - OPG/IL9R/cGC-F
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 98 - protein, 737 aa, synthetic construct
  REGION 1..737: note = MISC_FEATURE - OPG/IL9R/cGC-S
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 99 - protein, 525 aa, synthetic construct
  REGION 1..525: note = MISC_FEATURE - TACI/IL9R/cGC-F
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 100 - protein, 522 aa, synthetic construct
  REGION 1..522: note = MISC_FEATURE - TACI/IL9R/cGC-S
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 101 - protein, 414 aa, synthetic construct
  REGION 1..414: note = MISC_FEATURE - BCMA/IL9R/cGC-F
MLQMAGQCSQNEYFDSLLHACIPCQLRCSSNTPPLTCQRYCNASVTNSVKGTNALIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 102 - protein, 411 aa, synthetic construct
  REGION 1..411: note = MISC_FEATURE - BCMA/IL9R/cGC-S
MLQMAGQCSQNEYFDSLLHACIPCQLRCSSNTPPLTCQRYCNASVTNSVKGTNALIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 103 - protein, 582 aa, synthetic construct
  REGION 1..582: note = MISC_FEATURE - NGFR/IL9R/cGC-F
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 104 - protein, 579 aa, synthetic construct
  REGION 1..579: note = MISC_FEATURE - NGFR/IL9R/cGC-S
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 105 - protein, 521 aa, synthetic construct
  REGION 1..521: note = MISC_FEATURE - EDAR/IL9R/cGC-F
EYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATALIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 106 - protein, 518 aa, synthetic construct
  REGION 1..518: note = MISC_FEATURE - EDAR/IL9R/cGC-S
EYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATALIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 107 - protein, 516 aa, synthetic construct
  REGION 1..516: note = MISC_FEATURE - DCR2 (TNFRSF10D) /IL9R/cGC-F
ATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 108 - protein, 513 aa, synthetic construct
  REGION 1..513: note = MISC_FEATURE - DCR2 (TNFRSF10D) /IL9R/cGC-S
ATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 109 - protein, 571 aa, synthetic construct
  REGION 1..571: note = MISC_FEATURE - DCR1 (TNFRSF10C) /IL9R/cGC-F
ATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMITSPGTPALIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 110 - protein, 568 aa, synthetic construct
  REGION 1..568: note = MISC_FEATURE - DCR1 (TNFRSF10C) /IL9R/cGC-S
ATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMITSPGTPALIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 111 - protein, 533 aa, synthetic construct
  REGION 1..533: note = MISC_FEATURE - CD40/IL9R/cGC-F
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 112 - protein, 530 aa, synthetic construct
  REGION 1..530: note = MISC_FEATURE - CD40/IL9R/cGC-S
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 113 - protein, 508 aa, synthetic construct
  REGION 1..508: note = MISC_FEATURE - FAS/IL9R/cGC-F
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 114 - protein, 505 aa, synthetic construct
  REGION 1..505: note = MISC_FEATURE - FAS/IL9R/cGC-S
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 115 - protein, 576 aa, synthetic construct
  REGION 1..576: note = MISC_FEATURE - DR4/IL9R/cGC-F
ASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 116 - protein, 573 aa, synthetic construct
  REGION 1..573: note = MISC_FEATURE - DR4/IL9R/cGC-S
ASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 117 - protein, 668 aa, synthetic construct
  REGION 1..668: note = MISC_FEATURE - DR6/IL9R/cGC-F
QPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 118 - protein, 665 aa, synthetic construct
  REGION 1..665: note = MISC_FEATURE - DR6/IL9R/cGC-S
QPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 119 - protein, 515 aa, synthetic construct
  REGION 1..515: note = MISC_FEATURE - DR5/IL9R/cGC-F
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 120 - protein, 512 aa, synthetic construct
  REGION 1..512: note = MISC_FEATURE - DR5/IL9R/cGC-S
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 121 - protein, 535 aa, synthetic construct
  REGION 1..535: note = MISC_FEATURE - DR3/IL9R/cGC-F
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 122 - protein, 532 aa, synthetic construct
  REGION 1..532: note = MISC_FEATURE - DR3/IL9R/cGC-S
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 123 - protein, 595 aa, synthetic construct
  REGION 1..595: note = MISC_FEATURE - TNFRSF1B/IL9R/cGC-F
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 124 - protein, 592 aa, synthetic construct
  REGION 1..592: note = MISC_FEATURE - TNFRSF1B/IL9R/cGC-S
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 125 - protein, 542 aa, synthetic construct
  REGION 1..542: note = MISC_FEATURE - TNFRSF1/IL9R/cGC-F
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 126 - protein, 539 aa, synthetic construct
  REGION 1..539: note = MISC_FEATURE - TNFRSF1/IL9R/cGC-S
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 127 - protein, 473 aa, synthetic construct
  REGION 1..473: note = MISC_FEATURE - BMPR1B/IL9R/cGC-F
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 128 - protein, 470 aa, synthetic construct
  REGION 1..470: note = MISC_FEATURE - BMPR1B/IL9R/cGC-S
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 129 - protein, 489 aa, synthetic construct
  REGION 1..489: note = MISC_FEATURE - BMPR1A/IL9R/cGC-F
QNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 130 - protein, 486 aa, synthetic construct
  REGION 1..486: note = MISC_FEATURE - BMPR1A/IL9R/cGC-S
QNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 131 - protein, 484 aa, synthetic construct
  REGION 1..484: note = MISC_FEATURE - BMPR2/IL9R/cGC-F
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 132 - protein, 481 aa, synthetic construct
  REGION 1..481: note = MISC_FEATURE - BMPR2/IL9R/cGC-S
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 133 - protein, 963 aa, synthetic construct
  REGION 1..963: note = MISC_FEATURE - CSF3R/IL9R/cGC-F
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 134 - protein, 960 aa, synthetic construct
  REGION 1..960: note = MISC_FEATURE - CSF3R/IL9R/cGC-S
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 135 - protein, 858 aa, synthetic construct
  REGION 1..858: note = MISC_FEATURE - CSF1R/IL9R/cGC-F
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 136 - protein, 855 aa, synthetic construct
  REGION 1..855: note = MISC_FEATURE - CSF1R/IL9R/cGC-S
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 137 - protein, 1092 aa, synthetic construct
  REGION 1..1092: note = MISC_FEATURE - VEGF Receptor 1/IL9R/cGC-F
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 138 - protein, 1089 aa, synthetic construct
  REGION 1..1089: note = MISC_FEATURE - VEGF Receptor 1/IL9R/cGC-S
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 139 - protein, 1105 aa, synthetic construct
  REGION 1..1105: note = MISC_FEATURE - VEGF Receptor 2/IL9R/cGC-F
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 140 - protein, 1102 aa, synthetic construct
  REGION 1..1102: note = MISC_FEATURE - VEGF Receptor 2/IL9R/cGC-S
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 141 - protein, 1108 aa, synthetic construct
  REGION 1..1108: note = MISC_FEATURE - VEGF Receptor 3/IL9R/cGC-S
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 142 - protein, 1111 aa, synthetic construct
  REGION 1..1111: note = MISC_FEATURE - VEGF Receptor 3/IL9R/cGC-F
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 143 - protein, 463 aa, synthetic construct
  REGION 1..463: note = MISC_FEATURE - Activin R1A ectodomain/IL9R/cGC-F
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 144 - protein, 460 aa, synthetic construct
  REGION 1..460: note = MISC_FEATURE - Activin R1A ectodomain/IL9R/cGC-S
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 145 - protein, 463 aa, synthetic construct
  REGION 1..463: note = MISC_FEATURE - Activin R1B ectodomain/IL9R/cGC-F
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 146 - protein, 460 aa, synthetic construct
  REGION 1..460: note = MISC_FEATURE - Activin R1B ectodomain/IL9R/cGC-S
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 147 - protein, 452 aa, synthetic construct
  REGION 1..452: note = MISC_FEATURE - Activin R1C ectodomain/IL9R/cGC-F
LSPGLKCVCLLCDSSNFTCQTEGACWASVMLTNGKEQVIKSCVSLPELNAQVFCHSSNNVTKTECCFTDFCNNITLHLPTASPNAPKLGPMELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 148 - protein, 449 aa, synthetic construct
  REGION 1..449: note = MISC_FEATURE - Activin R1C ectodomain/IL9R/cGC-S
LSPGLKCVCLLCDSSNFTCQTEGACWASVMLTNGKEQVIKSCVSLPELNAQVFCHSSNNVTKTECCFTDFCNNITLHLPTASPNAPKLGPMELIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 149 - protein, 479 aa, synthetic construct
  REGION 1..479: note = MISC_FEATURE - Activin R2B ectodomain/IL9R/cGC-F
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 150 - protein, 476 aa, synthetic construct
  REGION 1..476: note = MISC_FEATURE - Activin R2B ectodomain/IL9R/cGC-S
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 151 - protein, 476 aa, synthetic construct
  REGION 1..476: note = MISC_FEATURE - Activin R2A ectodomain/IL9R/cGC-F
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 152 - protein, 473 aa, synthetic construct
  REGION 1..473: note = MISC_FEATURE - Activin R2A ectodomain/IL9R/cGC-S
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 153 - protein, 574 aa, synthetic construct
  REGION 1..574: note = MISC_FEATURE - IL10R-alpha/IL9R/cGC-F
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 154 - protein, 571 aa, synthetic construct
  REGION 1..571: note = MISC_FEATURE - IL10R-alpha/IL9R/cGC-S
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 155 - protein, 504 aa, synthetic construct
  REGION 1..504: note = MISC_FEATURE - TGFBR2 ectodomain/IL9R/cGC-F
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 156 - protein, 501 aa, synthetic construct
  REGION 1..501: note = MISC_FEATURE - TGFBR2 ectodomain/IL9R/cGC-S
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 157 - protein, 453 aa, synthetic construct
  REGION 1..453: note = MISC_FEATURE - TGFBR1 ectodomain/IL9R/cGC-F
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 158 - protein, 450 aa, synthetic construct
  REGION 1..450: note = MISC_FEATURE - TGFBR1 ectodomain/IL9R/cGC-S
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 159 - protein, 480 aa, synthetic construct
  REGION 1..480: note = MISC_FEATURE - TIGIT/IL9R/cGC-F
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 160 - protein, 477 aa, synthetic construct
  REGION 1..477: note = MISC_FEATURE - TIGIT/IL9R/cGC-S
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 161 - protein, 535 aa, synthetic construct
  REGION 1..535: note = MISC_FEATURE - FCGR2B/IL9R/cGC-F
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 162 - protein, 532 aa, synthetic construct
  REGION 1..532: note = MISC_FEATURE - FCGR2B/IL9R/cGC-S
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 163 - protein, 637 aa, synthetic construct
  REGION 1..637: note = MISC_FEATURE - FCGR1/IL9R/cGC-F
QVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 164 - protein, 634 aa, synthetic construct
  REGION 1..634: note = MISC_FEATURE - FCGR1/IL9R/cGC-S
QVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 165 - protein, 568 aa, synthetic construct
  REGION 1..568: note = MISC_FEATURE - 2B4/IL9R/cCG-F
CQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 166 - protein, 565 aa, synthetic construct
  REGION 1..565: note = MISC_FEATURE - 2B4/IL9R/cCG-S
CQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 167 - protein, 504 aa, synthetic construct
  REGION 1..504: note = MISC_FEATURE - LAIR1/IL9R/cGC-F
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 168 - protein, 501 aa, synthetic construct
  REGION 1..501: note = MISC_FEATURE - LAIR1/IL9R/cGC-S
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 169 - protein, 708 aa, synthetic construct
  REGION 1..708: note = MISC_FEATURE - CD5/IL-9R/cGC-F
RLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 170 - protein, 705 aa, synthetic construct
  REGION 1..705: note = MISC_FEATURE - CD5/IL-9R/cGC-S
RLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 171 - protein, 413 aa, synthetic construct
  REGION 1..413: note = MISC_FEATURE - TWEAKR/IL9R/cGC-F
EQAPGTAPCSRGSSWSADLDKCMDCASCRARPHSDFCLGCAAAPPAPFRLLWPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 172 - protein, 410 aa, synthetic construct
  REGION 1..410: note = MISC_FEATURE - TWEAKR/IL9R/cGC-S
EQAPGTAPCSRGSSWSADLDKCMDCASCRARPHSDFCLGCAAAPPAPFRLLWPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 173 - protein, 402 aa, synthetic construct
  REGION 1..402: note = MISC_FEATURE - TWEAKR/IL9R/cGC-F/TNFR1-TM
EQAPGTAPCSRGSSWSADLDKCMDCASCRARPHSDFCLGCAAAPPAPFRLLWPVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 174 - protein, 399 aa, synthetic construct
  REGION 1..399: note = MISC_FEATURE - TWEAKR/IL9R/cGC-S/TNFR1-TM
EQAPGTAPCSRGSSWSADLDKCMDCASCRARPHSDFCLGCAAAPPAPFRLLWPVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 175 - protein, 729 aa, synthetic construct
  REGION 1..729: note = MISC_FEATURE - OPG/IL9R/cGC-F/TNFR1-TM
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 176 - protein, 726 aa, synthetic construct
  REGION 1..726: note = MISC_FEATURE - OPG/IL9R/cGC-S/TNFR1-TM
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 177 - protein, 514 aa, synthetic construct
  REGION 1..514: note = MISC_FEATURE - TACI/IL9R/cGC-F/TNFR1-TM
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 178 - protein, 511 aa, synthetic construct
  REGION 1..511: note = MISC_FEATURE - TACI/IL9R/cGC-S/TNFR1-TM
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 179 - protein, 403 aa, synthetic construct
  REGION 1..403: note = MISC_FEATURE - BCMA/IL9R/cGC-F/TNFR1-TM
MLQMAGQCSQNEYFDSLLHACIPCQLRCSSNTPPLTCQRYCNASVTNSVKGTNAVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 180 - protein, 400 aa, synthetic construct
  REGION 1..400: note = MISC_FEATURE - BCMA/IL9R/cGC-S/TNFR1-TM
MLQMAGQCSQNEYFDSLLHACIPCQLRCSSNTPPLTCQRYCNASVTNSVKGTNAVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 181 - protein, 571 aa, synthetic construct
  REGION 1..571: note = MISC_FEATURE - NGFR/IL9R/cGC-F/TNFR1-TM
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 182 - protein, 568 aa, synthetic construct
  REGION 1..568: note = MISC_FEATURE - NGFR/IL9R/cGC-S/TNFR1-TM
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 183 - protein, 510 aa, synthetic construct
  REGION 1..510: note = MISC_FEATURE - EDAR/IL9R/cGC-F/TNFR1-TM
EYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATAVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 184 - protein, 507 aa, synthetic construct
  REGION 1..507: note = MISC_FEATURE - EDAR/IL9R/cGC-S/TNFR1-TM
EYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATAVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 185 - protein, 505 aa, synthetic construct
  REGION 1..505: note = MISC_FEATURE - DCR2 (TNFRSF10D) /IL9R/cGC-F/TNFR1-TM
ATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 186 - protein, 502 aa, synthetic construct
  REGION 1..502: note = MISC_FEATURE - DCR2 (TNFRSF10D) /IL9R/cGC-S/TNFR1-TM
ATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 187 - protein, 560 aa, synthetic construct
  REGION 1..560: note = MISC_FEATURE - DCR1 (TNFRSF10C) /IL9R/cGC-F/TNFR1-TM
ATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMITSPGTPAVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 188 - protein, 557 aa, synthetic construct
  REGION 1..557: note = MISC_FEATURE - DCR1 (TNFRSF10C) /IL9R/cGC-S/TNFR1-TM
ATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMITSPGTPAVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 189 - protein, 522 aa, synthetic construct
  REGION 1..522: note = MISC_FEATURE - CD40/IL9R/cGC-F/TNFR1-TM
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 190 - protein, 519 aa, synthetic construct
  REGION 1..519: note = MISC_FEATURE - CD40/IL9R/cGC-S/TNFR1-TM
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 191 - protein, 497 aa, synthetic construct
  REGION 1..497: note = MISC_FEATURE - FAS/IL9R/cGC-F/TNFR1-TM
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 192 - protein, 494 aa, synthetic construct
  REGION 1..494: note = MISC_FEATURE - FAS/IL9R/cGC-S/TNFR1-TM
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 193 - protein, 565 aa, synthetic construct
  REGION 1..565: note = MISC_FEATURE - DR4/IL9R/cGC-F/TNFR1-TM
ASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 194 - protein, 562 aa, synthetic construct
  REGION 1..562: note = MISC_FEATURE - DR4/IL9R/cGC-S/TNFR1-TM
ASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 195 - protein, 654 aa, synthetic construct
  REGION 1..654: note = MISC_FEATURE - DR6/IL9R/cGC-S/TNFR1-TM
QPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 196 - protein, 504 aa, synthetic construct
  REGION 1..504: note = MISC_FEATURE - DR5/IL9R/cGC-F/TNFR1-TM
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 197 - protein, 501 aa, synthetic construct
  REGION 1..501: note = MISC_FEATURE - DR5/IL9R/cGC-S/TNFR1-TM
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 198 - protein, 524 aa, synthetic construct
  REGION 1..524: note = MISC_FEATURE - DR3/IL9R/cGC-F/TNFR1-TM
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 199 - protein, 521 aa, synthetic construct
  REGION 1..521: note = MISC_FEATURE - DR3/IL9R/cGC-S/TNFR1-TM
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 200 - protein, 584 aa, synthetic construct
  REGION 1..584: note = MISC_FEATURE - TNFRSF1B/IL9R/cGC-F/TNFR1-TM
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 201 - protein, 581 aa, synthetic construct
  REGION 1..581: note = MISC_FEATURE - TNFRSF1B/IL9R/cGC-S/TNFR1-TM
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 202 - protein, 531 aa, synthetic construct
  REGION 1..531: note = MISC_FEATURE - TNFRSF1/IL9R/cGC-F/TNFR1-TM
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 203 - protein, 528 aa, synthetic construct
  REGION 1..528: note = MISC_FEATURE - TNFRSF1/IL9R/cGC-S/TNFR1-TM
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFQPQPQPQPQPQPERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 204 - protein, 489 aa, synthetic construct
  REGION 1..489: note = MISC_FEATURE - anti-HER2 CAR (4D5)
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 205 - protein, 657 aa, synthetic construct
  REGION 1..657: note = MISC_FEATURE - DR6/IL9R/cGC-F/TNFR1-TM
QPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHVLLPLVIFFGLCLLSLLFIGLMYVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFGGGGSGGGGSGGGGSERTMPRIPTLKNLEDLVTEYHGNFSAWSGVSKGLAESLQPDYSERLCLVSEIPPKGGALGEGPGASPCNQHSPYWAPPCYTLKPET